(19) United States
(12) Patent Application Publication
(10) Pub. No.: US 2008/0234318 A1
(43) Pub. Date: Sep. 25, 2008

(54) Chemical Compounds

(76) Inventors: Kristjan Gudmundsson, Durham, NC (US); Sharon Davis Boggs, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/065,089

(22) PCT Filed: Aug. 30, 2006
(86) PCT No.: PCT/US06/34195

§ 371 Date: May 9, 2008

Related U.S. Application Data
(60) Provisional application No. 60/713,134, filed on Aug. 31, 2005.

(51) Int. Cl. A61K 31/47; C07D 215/38

(52) U.S. Cl. 514/313; 546/162

(57) ABSTRACT

The present invention provides compounds that demonstrate protective effects on target cells from HIV infection in a manner as to bind to chemokine receptor, and which affect the binding of the natural ligand or chemokine to a receptor such as CXCR4 of a target cell.

### FIELD OF THE INVENTION

The present invention provides compounds that demonstrate protective effects on target cells from HIV infection in a manner as to bind to a chemokine receptor, and which affect the binding of the natural ligand or chemokine to a receptor such as CXCR4 of a target cell.

### BACKGROUND OF THE INVENTION

HIV gains entry into host cells by means of the CD4 receptor and at least one co-receptor expressed on the surface of the cell membrane. M-tropic strains of HIV utilize the chemokine receptor CCR5, whereas T-tropic strains of HIV mainly use CXCR4 as the co-receptor. HIV co-receptor usage largely depends on hyper-variable regions of the V3 loop located on the viral envelope protein gp120. Binding of gp120 with CD4 and the appropriate co-receptor results in a conformational change and unmasking of a second viral envelope protein called gp41. The protein gp41 subsequently interacts with the host cell membrane resulting in fusion of the viral envelop with the cell. Subsequent transfer of viral genetic information into the host cell allows for the continuation of viral replication. Thus infection of host cells with HIV is usually associated with the virus gaining entry into the cell via the formation of the ternary complex of CCR5 or CXCR4, CD4, and gp120.

A pharmacological agent that would inhibit the interaction of gpi 20 with either CCR5/CD4 or CXCR4/CD4 would be a useful therapeutic in the treatment of a disease, disorder, or condition characterized by infection with M-tropic or T-tropic strains, respectively, either alone or in combination therapy.

Evidence that administration of a selective CXCR4 antagonist could result in an effective therapy comes from in vitro studies that have demonstrated that addition of ligands selective for CXCR4 as well as CXCR4-neutralizing antibodies to cells can block HIV viral/host cell fusion. In addition, human studies with a selective CXCR4 antagonist, have demonstrated that such compounds can significantly reduce T-tropic HIV viral load in those patients that are either dual tropic or those where only the T-tropic form of the virus is present.

In addition to serving as a co-factor for HIV entry, it has been recently suggested that the direct interaction of the HIV viral protein gp120 with CXCR4 could be a possible cause of CD8⁺ T-cell apoptosis and AIDS-related dementia via induction of neuronal cell apoptosis.

The signal provided by SDF-1 on binding to CXCR4 may also play an important role in tumor cell proliferation and regulation of angiogenesis associated with tumor growth; the known angiogenic growth factors VEG-F and bFGF up-regulate levels of CXCR4 in endothelial cells and SDF-1 can induce neovascularization in vivo. In addition, leukemia cells that express CXCR4 migrate and adhere to lymph nodes and bone marrow stromal cells that express SDF-1.

The binding of SDF-1 to CXCR4 has also been implicated in the pathogenesis of atherosclerosis, renal allograft rejection asthma and allergic airway inflammation, Alzheimer's disease, and arthritis.

Additionally, CXCR4 antagonists may have a role in remodeling and repair of cardiac tissue and preserving cardiac function post myocardial infarction. After myocardial infarction, peripheral and bone marrow derived endothethial progenitor cells are found within the myocardium. It is thought that these cells result in improved ventricular function. This may be due to the production of cytokines that restore function and vascularization or to differentiation of the cells into functional myocardium. CXCL12 and CXCR4 are required for the homing of these stem cells to the myorcardium. In a preclinical study, a CXCR4 antagonist preserved chronic left ventricular function in rats after induction of a myocardial infarction by promoting mobilization and incorporation of bone marrow-derived enothethial progenitor cells into sites of myocardial neovascularization.

The present invention is directed to compounds that can act as agents that modulate chemokine receptor activity. Such chemokine receptors may include, but are not limited to, CCR1, CCR2, CCR3, CCR4, CCR5, CCR6, CCR7, CCR8, CXCR1, CXCR2, CXCR3, CXCR4, and CXCR5.

The present invention provides compounds that demonstrate protective effects on target cells from HIV infection in a manner as to bind to a chemokine receptor, and which affect the binding of the natural ligand or chemokine to a receptor, such as CXCR4 of a target cell.

### SUMMARY OF THE INVENTION

The present invention includes compounds of formula (I):

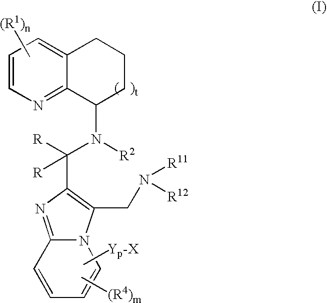

wherein:
t is 0, 1, or 2;
each R independently is H, C₁-C₈ alkyl, C₂-C₆ alkenyl, C₂-C₆ alkynyl, C₁-C₈ haloalkyl, C₃-C₈ cycloalkyl, —R^{a}Ay, —R¹⁰R¹⁰, or —R^{a}S(O)_{q}R¹⁰;
each R independently is halogen, C₁₋₈ haloalkyl, C₁-C₈ alkyl, C₂-C₆ alkenyl, C₂-C₆ alkynyl, C₃-C₈ cycloalkyl, C₃-C₈ cycloalkenyl, -Ay, —NHAy, -Het, —NHHet, —OR¹⁰, —OAy, —OHet, —R⁸OR¹⁰, —NR⁶R⁷, —R^{a}NR⁶R⁷, —R^{a}C(O)R¹⁰, —C(O)R¹⁰, —CO₂R¹⁰, —R²CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from the group consisting of H, C₁-C₈ alkyl, C₁-C₈ haloalkyl, C₃-C₈ cycloalkyl, C₂-C₈ alkenyl, C₂-C₈ alkynyl, —R^{a}Ay, —R^{a}cycloalkyl, —R^{a}OR⁵, and —R^{a}S(O)_{q}R⁵;

R¹¹ and R¹² are independently selected from the group consisting of H, C₁-C₈alkyl, C₁-C₈ haloalkyl, C₂-C₈ alkenyl, C₂-C₆ alkynyl, C₃-C₈cycloalkyl, C₃-C₈ cycloalkenyl, -Ay, -Het, R^{a}OR¹⁰, R^{a}NR⁶R⁷, —R^{a}C(O)R¹⁰, —C(O)R¹⁰, —C(O)R^{a}Ay, —CO₂R(, —CO₂R^{a}Ay, —R^{a}CO₂R¹⁰, —C(O)NR⁶R⁷, —C(O)Ay, —C(O)Het, —S(O)₂NR⁶R⁷, S(O)_{q}R¹⁰, —S(O)_{q}R^{a}Ay, —S(O)_{q}Ay, and —S(O)_{q}Het; or R¹¹ and R¹² link to form a heterocyclic ring optionally substituted with one or more of C₁-C₈ alkyl, C₂-C₈ alkenyl, C₂-C₈ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈-alkylamino;

each R⁴ independently is halogen, C₁-C₈ haloalkyl, C₁-C₈alkyl, C₂-C₆ alkenyl, C₂-C₆ alkynyl, C₃-C₈ cycloalkyl, C₃-C₈cycloalkenyl, -Ay, —NHAY, -Het, —NHHet, —OR¹⁰, —OAy, -OHet, —R^{a}OR¹⁰, —NR⁶R⁷, —R^{a}NR⁶R⁷, —ROC(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, —S(O)_{q}Het, cyano, nitro, or azido;
m is 0, 1, or 2;
each R⁵ independently is H, C₁-C₈ alkyl, C₂-C₆ alkenyl, C₂-C₆ alkynyl, C₃-C₈ cycloalkyl, or -Ay;
p is 0 or 1;
Y is —NR¹⁰—, —O—, —C(O)NR¹⁰—, —NR¹⁰C(O)—, —C(O)—, —C(O)O—, —NR¹⁰C(O)N(R¹⁰)—, —S(O)_{q}—, —S(O)_{q}NR¹⁰, or —NR¹⁰S(O)_{q}—;
X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is C₁-C₈ alkylene, C₃-C₈ cycloalkylene, C₂-C₆ alkenylene, C₃-C₈ cycloalkenylene, or C₂-C₆ alkynylene, optionally substituted with one or more C₁-C₈ alkyl, hydroxyl or oxo;
each R¹⁰ is independently H, C₁-C₈ alkyl, C₃-C₈ cycloalkyl, C₂-C₆ alkenyl, C₂-C₆ alkynyl, C₃-C₈ cycloalkenyl, —R^{a}cycloalkyl, —R^{a}OH, —R^{a}OR⁵, —R^{a}NR⁶R⁷, or —R^{a}Het
each of R⁶ and R⁷ independently is selected from H, C₁-C₈ alkyl, C₁-C₈ alkenyl, C₂-C₆ alkynyl, C₃-C₈ cycloalkyl, C₃-C₈ cycloalkenyl, —R^{a}cycloalkyl, —R^{a}OH, —R^{a}OR⁵, —R^{a}NR⁸R⁹, -Ay, -Het, —R^{a}Ay, —R^{a}Het, or —S(O)_{q}R⁵;
each of R⁸ and R⁹ is independently selected from H or C₁-C₈ alkyl;
each q independently is 0, 1, or 2;
each Ay independently represents a C₄-C₁₄ aryl group optionally substituted with one or more of C₁-C₈ alkyl, C₂-C₈ alkenyl, C₂-C₈ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; and
each Het independently represents a C₃-C₁₁ heterocyclyl or heteroaryl group optionally substituted with one or more of C₁-C₈ alkyl, C₂-C₆ alkenyl, C₂-C₆ alkynyl, C₁-C₈alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; or pharmaceutically acceptable derivatives thereof.

The present invention features a compound of formula (I) wherein, t is 1 or 2 and all other substituents are as defined above or a pharmaceutically acceptable derivative thereof. The present invention features a compound of formula (I) wherein t is 1 and all other substituents are as defined above or a pharmaceutically acceptable derivative thereof.

The present invention features a compound of formula (I) wherein each R is H or alkyl and all other substituents are as defined above or a pharmaceutically acceptable derivative thereof. The present invention also features a compound of formula (I) wherein each R is H.

The present invention features a compound of formula (I) wherein n is 0 and all other substituents are as defined above or a pharmaceutically acceptable derivative thereof.

The present invention features a compound of formula (I) wherein n is 1 and R¹ is halogen, C₁-C₈ haloalkyl, C₁-C₈ alkyl, OR¹⁰, NR⁶R⁷, CO₂R¹⁰, CONR⁶R⁷, or cyano and all other substituents are as defined above or a pharmaceutically acceptable derivative thereof.

The present invention features a compound of formula (I) wherein R² is H, C₁-C₈alkyl, C₁-C₈haloalkyl, —R^{a}cycloalkyl, R^{a}OR⁵, or C₃-C₈cycloalkyl and all other substituents are as defined above or a pharmaceutically acceptable derivative thereof. The present invention features a compound of formula (I) wherein R² is C₁-C₈alkyl, RaOR⁵ or C₃-C₈cycloalkyl. The present invention features a compound of formula (I) wherein R² is C₁-C₈alkyl.

The present invention features a compound of formula (I) wherein m is 0 and all other substituents are as defined above or a pharmaceutically acceptable derivative thereof.

The present invention features a compound of formula (I) wherein m is 1 or 2 and R⁴ is one or more of halogen, C₁-C₈haloalkyl, C₁-C₈alkyl, OR¹⁰, NR⁶R⁷, CO₂R¹⁰, CONR⁶R⁷, or cyano and all other substituents are as defined above or a pharmaceutically acceptable derivative thereof. The present invention features a compound of formula (I) wherein m is 1 and all other substituents are as defined above or a pharmaceutically acceptable derivative thereof.

The present invention features a compound of formula (I) wherein R⁴ is one or more of halogen, C₁-C₈haloalkyl, C₁-C₈alkyl, OR¹⁰, NR⁶R⁷, CO₂R¹⁰, CONR⁶R⁷, or cyano and all other substituents are as defined above or a pharmaceutically acceptable derivative thereof.

The present invention features a compound of formula (I) wherein p is 0 and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂ and all other substituents are as defined above or a pharmaceutically acceptable derivative thereof. The present invention features a compound of formula (I) wherein X is —R^{a}N(R¹⁰)₂, -Het, —R^{a}Het, -HetN(R¹⁰)₂, —R^{a}HetN(R¹⁰)₂, or -HetR^{a}N(R¹⁰)₂ and all other substituents are as defined above or a pharmaceutically acceptable derivative thereof. The present invention features a compound of formula (I) wherein X is R^{a}N(R¹⁰)₂, -Het, —R^{a}Het, or -HetN(R¹⁰)₂ and all other substituents are as defined above or a pharmaceutically acceptable derivative thereof.

The present invention features a compound of formula (I) wherein p is 1; Y is —N(R¹⁰)—, —O—, —S—, —CONR¹⁰—, —NR¹⁰CO—, or —S(O)_{q}NR¹⁰—; and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂ and all other substituents are as defined above or a pharmaceutically acceptable derivative thereof. The present invention features a compound of formula (I) wherein Y is —N(R¹⁰)—, —O—, —CONR¹⁰—, —NR¹⁰CO— and X is —R^{a}N(R¹⁰)₂, -Het, —R^{a}Het, or -HetN(R¹⁰)₂ and all other substituents are as defined above or a pharmaceutically acceptable derivative thereof,

The present invention features a compound of formula (I) wherein Het is piperidine, piperazine, azetidine, pyrrolidine, imidazole, pyridine, and the like and all other substituents are as defined above or a pharmaceutically acceptable derivative thereof.

The present invention features a compound of formula (I) wherein, each R is H and t is 1 and all other substituents are as defined above or a pharmaceutically acceptable derivative thereof.

The present invention features a compound of formula (I) wherein p is 0 and X is -Het and all other substituents are as defined above or a pharmaceutically acceptable derivative thereof. The present invention features a compound of formula (I) wherein -Het is unsubstituted or substituted with one or more C₁-C₈ alkyl or C₃-C₈ cycloalkyl and all other substituents are as defined above or a pharmaceutically acceptable derivative thereof. The present invention features a compound of formula (I) wherein -Het is piperazine or C₁-C₈ alkyl substituted piperazine and all other substituents are as defined above or a pharmaceutically acceptable derivative thereof.

The present invention features a compound of formula (I) wherein R¹¹ and R¹² are independently selected from a group consisting of H, C₁-C₈ alkyl, C₁-C₈ haloalkyl, C₂-C₆alkenyl, C₂-C₆ alkynyl, C₃-C₈cycloalkyl, C₃-C₈ cycloalkenyl, -Ay, -Het, —R^{a}OR¹⁰, —R^{a}NR⁶R⁷, —R^{a}C(O)R¹⁰, —C(O)R¹⁰, —C(O)R^{a}Ay, —CO₂R¹⁰, —CO₂R^{a}Ay, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}R^{a}Ay, —S(O) y, and —S(O)_{q}Het; or R¹¹ and R¹² link to form a heterocyclic ring optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈-cycloalkoxy, cyano, amide, amino, and C₁-C₈ alkylamino; and all other substituents are as defined above with respect to formula (I).

The present invention features a compound of formula (I) wherein, the substituent —Yₚ—X is located on the depicted imidazopyridine ring as in formula (I-A):

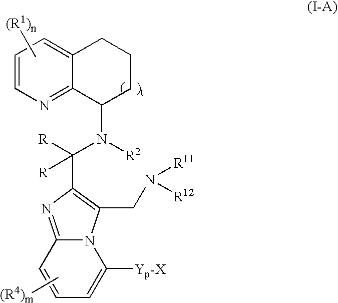

wherein all substituents are as defined above, or pharmaceutically acceptable derivatives thereof.

One aspect of the invention includes compounds of formula (I-A) wherein p is 0, X is -Het, and -Het is piperazine or C₁-C₈ alkyl substituted piperazine, or pharmaceutically acceptable derivatives thereof.

One aspect of the present invention features a compound of formula (I-A) wherein t is 1, n is 0, each R is H, R² is C₁-C₈ alkyl, R¹¹ and R¹² link together to form a heterocyclic ring optionally substituted with one or more of C₁-C₈ alkyl, C₂-C₆ alkenyl, C₂-C₆ alkynyl, C₁-C₈alkoxy, hydroxyl, halogen, C₁-C₈ haloalkyl, C₃-C₈ cycloalkyl, C₃-C₈ cycloalkoxy, cyano, amide, amino, and C₁-C₈ alkylamino, m is 0, p 0 and X is -Het, optionally substituted with C₁-C₈ alkyl, C₂-C₆ alkenyl, C₂-C₆ alkynyl, C₁-C₈alkoxy, hydroxyl, halogen, C₃-C₈ haloalkyl, C₃-C₈ cycloalkyl, C₃-C₈ cycloalkoxy, cyano, amide, amino, and C₁-C₈ alkylamino, or pharmaceutically acceptable derivatives thereof.

One aspect of the present invention features a compound of formula I-A wherein t is 1, n is 0, each R is H, R² is C₁-C₈ alkyl, one of R¹¹ or R¹² is C(O)NR⁶R⁷; C(O)₂R¹⁰; C(O)R¹⁰, S(O)₂NR⁶R⁷ and S(O)_{q}R¹⁰ and one of R¹¹ or R¹² is H or C₁-C₈ alkyl, m is 0, p is 0 and X is -Het, optionally substituted with C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino, or pharmaceutically acceptable derivatives thereof.

One aspect of the present invention features a compound of formula I-A wherein t is 1, n is 0, each R is H, R² is C₁-C₈ alkyl or R^{a}cycloalkyl, one of R¹¹ or R¹² is C(O)NR⁶R⁷; C(O)₂R¹⁰; C(O)R¹⁰, S(O)₂NR⁶R⁷ and S(O)_{q}R¹⁰ and one of R¹¹ or R¹² is H or C₁-C₈ alkyl, m is 0, p is 0 and X is -Het, optionally substituted with C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino, or pharmaceutically acceptable derivatives thereof.

One aspect of the present invention features a compound of formula I-A wherein t is 1, n is 0, each R is H, R² is C₁-C₈ alkyl, one of R¹¹ or R¹² is C(O)NR⁶R⁷; C(O)₂R¹⁰; C(O)R¹⁰, S(O)₂NR⁶R⁷ and S(O)_{q}R¹⁰ and one of R¹¹ or R¹² is H or C₁-C₈ alkyl, m is 0, p is 0 and X is -HetN(R¹⁰)₂ wherein R¹⁰ is H or C₁-C₈ alkyl, or pharmaceutically acceptable derivatives thereof.

Compounds of the present invention include:
(8S)—N-{[3-[(Dimethylamino)methyl]-5-(4-methyl-1-piperazinyl)imidazo[1,2-a]pyridin-2-yl]methyl}-N-methyl-5,6,7,8-tetrahydro-8-quinolinamine;
(8S)—N-{[3-[(Diethylamino)methyl]-5-(4-methyl-1-piperazinyl)imidazo[1,2-a]pyridin-2-yl]methyl}-N-methyl-5,6,7,8-tetrahydro-8-quinolinamine;
(8S)—N-Methyl-N-{[5-(4-methyl-1-piperazinyl)-3-(1-pyrrolidinylmethyl)imidazo[1,2-a]pyridin-2-yl]methyl}-5,6,7,8-tetrahydro-8-quinolinamine;
2,2′-({[5-(4-Methyl-1-piperazinyl)-2-({methyl[(8S)-5,6,7,8-tetrahydro-8-quinolinyl]amino}methyl)imidazo[1,2-a]pyridin-3-yl]methyl}imino)diethanol;
2-(Ethyl{[5-(4-methyl-1-piperazinyl)-2-({methyl[(8S)-5,6,7,8-tetrahydro-8-quinolinyl]amino}methyl)imidazo[1,2-a]pyridin-3-yl]methyl}amino)ethanol;
1-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-3-pyrrolidinol;
(8S)—N-Methyl-N-{[5-(4-methyl-1-piperazinyl)-3-(4-morpholinylmethyl)imidazo[1,2-a]pyridin-2-yl]methyl}-5,6,7,8-tetrahydro-8-quinolinamine;
(8S)—N-Methyl-N-{[3-[(methylamino)methyl]-5-(4-methyl-1-piperazinyl)imidazo[1,2-a]pyridin-2-yl]methyl}-5,6,7,8-tetrahydro-8-quinolinamine; and
(8S)—N-{[3-[(Ethylamino)methyl]-5-(4-methyl-1-piperazinyl)imidazo[1,2-a]pyridin-2-yl]methyl}-N-methyl-5,6,7,8-tetrahydro-8-quinolinamine;
(8S)—N-Methyl-N-({5-(4-methyl-1-piperazinyl)-3-[(4-methyl-1-piperazinyl)methyl]imidazo[1,2-a]pyridin-2-yl}methyl)-5,6,7,8-tetrahydro-8-quinolinamine;
(8S)—N-Methyl-N-{[5-(4-methyl-1-piperazinyl)-3-(1-piperidinylmethyl)imidazo[1,2-a]pyridin-2-yl]methyl}-5,6,7,8-tetrahydro-8-quinolinamine;
(8S)—N-Methyl-N-{[3-{[(1-methylethyl)amino]methyl}-5-(4-methyl-1-piperazinyl)imidazo[1,2-a]pyridin-2-yl]methyl}5,6,7,8-tetrahydro-8-quinolinamine;
(8S)—N-{[3-(Aminomethyl)-5-(4-methyl-1-piperazinyl)imidazo[1,2-a]pyridin-2-yl]methyl}-N-methyl-5,6,7,8-tetrahydro-8-quinolinamine;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}acetamide;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}propanamide;
2-Methyl-N-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}propanamide;
2-Methyl-N-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}butanamide;
2 N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}benzamide;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-2-phenylacetamide;
Methyl {[5-(4-methyl-1-piperazinyl)-2-({methyl[(8S)-5,6,7,8-tetrahydro-8-quinolinyl]amino}methyl)imidazo[1,2-a]pyridin-3-yl]methyl}carbamate;
Ethyl {[5-(4-methyl-1-piperazinyl)-2-({methyl[(8S)-5,6,7,8-tetrahydro-8-quinolinyl]amino}methyl)imidazo[1,2-a]pyridin-3-yl]methyl}carbamate;
Phenylmethyl {[5-(4-methyl-1-piperazinyl)-2-({methyl[(8S)-5,6,7,8-tetrahydro-8-quinolinyl]amino}methyl)imidazo[1,2-a]pyridin-3-yl]methyl}carbamate;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}methanesulfonamide;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}ethanesulfonamide;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-2-propanesulfonamide;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}benzenesulfonamide;
N-{[5-(4-Methyl-1-piperazinyl)-2-({methyl[(8S-5,6,7,8-tetrahydro-8-quinolinyl]amino}methyl)imidazo[1,2-a]pyridin-3-yl]methyl}-1-phenylmethanesulfonamide;
N-Ethyl-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea;
N-(1-Methylethyl)-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-N′-phenylurea;
N-[4-(Dimethylamino)phenyl]-N′-([5-(4-methyl-1-piperazinyl)-2-({methyl[(8S)-5,6,7,8-tetrahydro-8-quinolinyl]amino}methyl)imidazo[1,2-a]pyridin-3-yl]methyl}urea;
N-[4-(Methyloxy)phenyl]-N-{([5-(4-methyl-1-piperazinyl)-2-({methyl[(8S)-5,6,7,8-tetrahydro-8-quinolinyl]amino}methyl)imidazo[1,2-a]pyridin-3-yl]methyl}urea;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}urea; and pharmaceutically acceptable derivatives thereof.

One aspect of the present invention includes the compounds substantially as hereinbefore defined with reference to any one of the Examples.

One aspect of the present invention includes a pharmaceutical composition comprising one or more compounds of the present invention and a pharmaceutically acceptable carrier.

One aspect of the present invention includes one or more compounds of the present invention for use as an active therapeutic substance.

One aspect of the present invention includes one or more compounds of the present invention for use in the treatment or prophylaxis of diseases and conditions caused by inappropriate activity of CXCR4.

One aspect of the present invention includes one or more compounds of the present invention for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. In one embodiment the condition or disease is HIV infection, rheumatoid arthritis, inflammation, or cancer. In yet another embodiment the disease is HIV infection.

One aspect of the present invention includes the use of one or more compounds of the present invention in the manufacture of a medicament for use in the treatment or prophylaxis of a condition or disease modulated by a chemokine receptor. Preferably the chemokine receptor is CXCR4.

One aspect of the present invention includes use of one or more compounds of the present invention in the manufacture of a medicament for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. Preferably the use relates to a medicament wherein the condition or disorder is HIV infection, rheumatoid arthritis, inflammation, or cancer.

One aspect of the present invention includes a method for the treatment or prophylaxis of a condition or disease modulated by a chemokine receptor comprising the administration of one or more compounds of the present invention. Preferably the chemokine receptor is CXCR4.

One aspect of the present invention includes a method for the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers comprising the administration of one or more compounds of the present invention.

One aspect of the present invention includes a method for the treatment or prophylaxis of HIV infection, rheumatoid arthritis, inflammation, or cancer comprising the administration of one or more compounds of the present invention. One aspect of the invention includes a method for the treatment or prophylaxis of HIV infection.

### DETAILED DESCRIPTION OF THE INVENTION

Terms are used within their accepted meanings. The following definitions are meant to clarify, but not limit, the terms defined.

As used herein the term “alkyl” alone or in combination with any other term, refers to a straight or branched chain hydrocarbon, containing from one to twelve carbon atoms, unless specified otherwise. Examples of “alkyl” as used herein include, but are not limited to, methyl, ethyl, propyl, isopropyl, isobutyl, n-butyl, tert-butyl, sec-butyl, isopentyl, n-pentyl, n-hexyl, and the like.

As used throughout this specification, the preferred number of atoms, such as carbon atoms, will be represented by, for example, the phrase “Cₓ-C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds. Examples include, but are not limited to, vinyl, allyl, and the like.

As used herein the term “alkynyl” refers to a straight or branched chain aliphatic hydrocarbon containing one or more carbon-to-carbon triple bonds, which may occur at any stable point along the chain. Examples include, but are not limited to, ethynyl, propynyl, butynyl, pentynyl, and the like.

As used herein, the term “alkylene” refers to an optionally substituted straight or branched chain divalent hydrocarbon radical, preferably having from one to ten carbon atoms, unless specified otherwise. Examples of “alkylene” as used herein include, but are not limited to, methylene, ethylene, n-propylene, n-butylene, and the like. Preferred substituent groups include C₁-C₈ alkyl, hydroxyl or oxo.

As used herein, the term “alkenylene” refers to a straight or branched chain divalent hydrocarbon radical, preferably having from two to ten carbon atoms, unless specified otherwise, containing one or more carbon-to-carbon double bonds. Examples include, but are not limited to, vinylene, allylene or 2-propenylene, and the like.

As used herein, the term “alkynylene” refers to a straight or branched chain divalent hydrocarbon radical, preferably having from two to ten carbon atoms, unless otherwise specified, containing one or more carbon-to-carbon triple bonds. Examples include, but are not limited to, ethynylene and the like.

As used herein, the term “cycloalkyl” refers to an optionally substituted non-aromatic cyclic hydrocarbon ring. Unless otherwise indicated, cycloalkyl is composed of three to eight carbon atoms, Exemplary “cycloalkyl” groups include, but are not limited to, cyclopropyl, cyclobutyl, cyclopentyl, cyclohexyl, and cycloheptyl. As used herein, the term “cycloalkyl” includes an optionally substituted fused polycyclic hydrocarbon saturated ring and aromatic ring system, namely polycyclic hydrocarbons with less than maximum number of non-cumulative double bonds, for example where a saturated hydrocarbon ring (such as a cyclopentyl ring) is fused with an aromatic ring (herein “aryl,” such as a benzene ring) to form, for example, groups such as indane. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈cycloalkoxy, cyano, amide, amino, and C₁-C₈ alkylamino.

As used herein, the term “cycloalkenyl” refers to an optionally substituted non-aromatic cyclic hydrocarbon ring containing one or more carbon-to-carbon double bonds which optionally includes an alkylene linker through which the cycloalkenyl may be attached. Exemplary “cycloalkenyl” groups include, but are not limited to, cyclopropenyl, cyclobutenyl, cyclopentenyl, cyclohexenyl, and cycloheptenyl. Preferred substituent groups include C₁-C₈ alkyl, C₂-C₈ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈alkylamino.

As used herein, the term “cycloalkylene” refers to a divalent, optionally substituted non-aromatic cyclic hydrocarbon ring. Exemplary “cycloalkylene” groups include, but are not limited to, cyclopropylene, cyclobutylene, cyclopentylene, cyclohexylene, and cycloheptylene. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “cycloalkenylene” refers to a divalent optionally substituted non-aromatic cyclic hydrocarbon ring containing one or more carbon-to-carbon double bonds. Exemplary “cycloalkenylene” groups include, but are not limited to, cyclopropenylene, cyclobutenylene, cyclopentenylene, cyclohexenylene, and cycloheptenylene. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “heterocycle”, “heterocyclic” or “heterocyclyl” refers to an optionally substituted mono- or polycyclic ring system containing one or more degrees of unsaturation and also containing one or more heteroatoms. Preferred heteroatoms include N, O, and/or S, including N-oxides, sulfur oxides, and dioxides. More preferably, the heteroatom is N.

Preferably the heterocyclyl ring is three to twelve-membered, unless otherwise indicated, and is either fully saturated or has one or more degrees of unsaturation. Such rings may be optionally fused to one or more of another “heterocyclic” ring(s) or cycloalkyl ring(s). Examples of “heterocyclic” groups include, but are not limited to, tetrahydrofuran, pyran, 1,4-dioxane, 1,3-dioxane, piperidine, piperazine, pyrrolidine, morpholine, tetrahydrothiopyran, aziridine, azetidine and tetrahydrothiophene. When the heterocyclic ring has substituents, it is understood that the substituents may be attached to any atom in the ring, whether a heteroatom or a carbon atom, provided that a stable chemical structure results. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “aryl” refers to an optionally substituted carbocyclic aromatic moiety (such as phenyl or naphthyl) containing the specified number of carbon atoms, preferably 6-14 carbon atoms or 6-10 carbon atoms. The term aryl also refers to optionally substituted ring systems, for example anthracene, phenanthrene, or naphthalene ring systems. Examples of “aryl” groups include, but are not limited to, phenyl, naphthyl, indenyl, azulenyl, fluorenyl, anthracenyl, phenanthrenyl, tetrahydronaphthyl, indanyl, phenathridinyl, and the like. Unless otherwise indicated, the term aryl also includes each possible positional isomer of an aromatic hydrocarbon radical, such as 1-naphthyl, 2-naphthyl, 5-tetrahydronaphthyl, 6-tetrahydronaphthyl, 1 phenanthridinyl, 2-phenanthridinyl, 3-phenanthridinyl, 4-phenanthridinyl, and the like. Preferred substituent groups include C₁-C₈ alkyl, C₂-C₆ alkenyl, C₂-C₈ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino.

As used herein, the term “heteroaryl” refers to an optionally substituted monocyclic five to seven membered aromatic ring unless otherwise specified, or to an optionally substituted fused bicyclic aromatic ring system comprising two of such aromatic rings. These heteroaryl rings contain one or more nitrogen, sulfur, and/or oxygen atoms, where N-oxides, sulfur oxides, and dioxides are permissible heteroatom substitutions. Preferably, the heteroatom is N.

Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, benzimidizolyl, imidazopyridinyl, pyrazolopyridinyl, and pyrazolopyrimidinyl. Preferred substituent groups include C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and alkylamino.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents as perfluoroalkyl groups and the like.

As used herein the term “alkoxy” refers to a group —OR′, where R′ is alkyl as defined. Examples of suitable alkoxy radicals include, but are not limited to, methoxy, ethoxy, propoxy, isopropoxy, n-butoxy, isobutoxy, sec-butoxy, tert-butoxy, and the like.

As used herein the term “cycloalkoxy” refers to a group —OR′, where R′ is cycloalkyl as defined.

As used herein the term “alkoxycarbonyl” refers to groups such as:

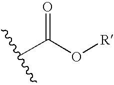

where the R′ represents an alkyl group as herein defined.

As used herein the term “aryloxycarbonyl” refers to groups such as:

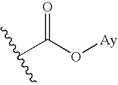

where the Ay represents an aryl group as herein defined.

As used herein the term “nitro” refers to a group —NO₂.

As used herein the term “cyano” refers to a group —CN.

As used herein the term “azido” refers to a group —N₃.

As used herein the term amino refers to a group —NR′R″, where R′ and R″ independently represent H, alkyl, alkenyl, alkynyl, cycloalkyl, heterocyclyl, aryl, or heteroaryl. Similarly, the term “alkylamino” includes an alkylene linker through which the amino group is attached.

As used herein the term “amide” refers to a group —C(O)NR′R″, where R′ and R″ independently represent H, alkyl, alkenyl, alkynyl, cycloalkyl, heterocyclyl, aryl, or heteroaryl.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted specifically. Rather, those of ordinary skill in the art will appreciate that the phrase is included to provide for modifications, which are encompassed within the scope of the appended claims.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically and/or diastereomerically enriched mixtures. Also included within the scope of the invention are the individual isomers of the compounds of the present invention, as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of the present invention, or a salt or other pharmaceutically acceptable derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

A “pharmaceutically acceptable derivative” means any pharmaceutically acceptable salt, ester, salt of an ester, ether, or other derivative of a compound of this invention which, upon administration to a recipient, is capable of providing directly or indirectly a compound of this invention or an inhibitorily active metabolite or residue thereof. Particularly favored derivatives and prodrugs are those that increase the bioavailability of the compounds of this invention when such compounds are administered to a mammal, for example, by allowing an orally administered compound to be more readily absorbed into the blood, or which enhance delivery of the parent compound to a biological compartment, for example, the brain or lymphatic system, relative to the parent species.

As used herein, the term “effective amount” means that amount of a drug or pharmaceutical agent that will elicit the biological or medical response of a tissue, system, animal, or human that is being sought, for instance, by a researcher or clinician. The term therapeutically “effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “modulators” as used herein is intended to encompass antagonist, agonist, inverse agonist, partial agonist or partial antagonist, inhibitors and activators.

In one aspect of the present invention, the compounds demonstrate protective effects against HIV infection by inhibiting binding of HIV to a chemokine receptor such as CXCR4 of a target cell. The invention includes a method that comprises contacting the target cell with an amount of the compound that is effective at inhibiting the binding of the virus to the chemokine receptor.

In addition to the role chemokine receptors play in HIV infection this receptor class has also been implicated in a wide variety of diseases. Thus CXCR4 modulators may also have a therapeutic role in the treatment of diseases associated with hematopoiesis, including but not limited to, controlling the side effects of chemotherapy, enhancing the success of bone marrow transplantation, enhancing wound healing and burn treatment, as well as combating bacterial infections in leukemia. In addition, compounds may also have a therapeutic role in diseases associated with inflammation, including but not limited to inflammatory or allergic diseases such as asthma, allergic rhinitis, hypersensitivity lung diseases, hypersensitivity pneumonitis, eosinophilic pneumonitis, delayed-type hypersensitivity, interstitial lung disease (ILD) (e.g. idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis), systemic anaphylaxis or hypersensitivity responses, drug allergies, insect sting allergies, autoimmune diseases such as rheumatoid arthritis, psoriatic arthritis, systemic lupus erythematosus, myastenia gravis, juvenile onset diabetes, glomerulonephritis, autoimmune throiditis, graft rejection, including allograft rejection or graft-versus-host disease, inflammatory bowel diseases, such as Crohn's disease and ulcerative colitus, spondyloarthropathies, scleroderma, psoriasis (including T-cell-mediated psoriasis) and inflammatory dermatoses such as dermatitis, eczema, atopic dermatitis, allergic contact dermatitis, urticaria, vasculitis (e.g. necrotizing, cutaneous, and hypersensitivity vasculitis), eoosinophilic myotis, eosinophilic fasciltis, and cancers.

The compounds according to the invention may also be used in adjuvant therapy in the treatment of HIV infections or HIV-associated symptoms or effects, for example Kaposi's sarcoma.

The present invention further provides a method for the treatment of a clinical condition in a patient, for example, a mammal including a human which clinical condition includes those which have been discussed hereinbefore, which comprises treating said patient with a pharmaceutically effective amount of a compound according to the invention. The present invention also includes a method for the treatment or prophylaxis of any of the aforementioned diseases or conditions.

Reference herein to treatment extends to prophylaxis as well as the treatment of established conditions, disorders and infections, symptoms thereof, and associated clinical conditions. The above compounds according to the invention and their pharmaceutically acceptable derivatives may be employed in combination with other therapeutic agents for the treatment of the above infections or conditions. Combination therapies according to the present invention comprise the administration of a compound of the present invention or a pharmaceutically acceptable derivative thereof and another pharmaceutically active agent. The active ingredient(s) and pharmaceutically active agents may be administered simultaneously (i.e., concurrently) in either the same or different pharmaceutical compositions or sequentially in any order. The amounts of the active ingredient(s) and pharmaceutically active agent(s) and the relative timings of administration will be selected in order to achieve the desired combined therapeutic effect.

For use in therapy, therapeutically effective amounts of a compound of the present invention, as well as salts, solvates, or other pharmaceutically acceptable derivatives thereof, may be administered as the raw chemical. Additionally, the active ingredient may be presented as a pharmaceutical composition.

Accordingly, the invention further provides pharmaceutical compositions that include effective amounts of compounds of the present invention and salts, solvates, or other pharmaceutically acceptable derivatives thereof, and one or more pharmaceutically acceptable carriers, diluents, or excipients. The compounds of the present invention and salts, solvates, or other pharmaceutically acceptable derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the present invention or salts, solvates, or other pharmaceutically acceptable derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of the present invention for the treatment of humans suffering from frailty, generally, should be in the range of 0.1 to 100 mg/kg body weight of recipient (mammal) per day. More usually the effective amount should be in the range of 0.1 to 10 mg/kg body weight per day. Thus, for, a 70 kg adult mammal one example of an actual amount per day would usually be from 7 to 700 mg. This amount may be given in a single dose per day or in a number (such as two, three, four, five, or more) of sub-doses per day such that the total daily dose is the same. An effective amount of a salt, solvate, or other pharmaceutically acceptable derivative thereof, may be determined as a proportion of the effective amount of the compound of the present invention per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the invention, with regard to certain conditions and disorders for which the compounds of the present invention are believed useful certain routes will be preferable to others.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of the present invention and salts, solvates, or other pharmaceutically acceptable derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of the present invention and salts, solvates, or other pharmaceutically acceptable derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by lontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, or other pharmaceutically acceptable derivatives thereof, may be employed alone or in combination with other therapeutic agents. The compound(s) of the present invention and the other pharmaceutically active agent(s) may be administered together or separately and, when administered separately, administration may occur simultaneously or sequentially, in any order. The amounts of the compound(s) of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds of the present invention may be used in the treatment of a variety of disorders and conditions and, as such, the compounds of the present invention may be used in combination with a variety of other suitable therapeutic agents useful in the treatment or prophylaxis of those disorders or conditions. The compounds may be used in combination with any other pharmaceutical composition where such combined therapy may be useful to modulate chemokine receptor activity and thereby prevent and treat inflammatory and/or immunoregulatory diseases.

The present invention may be used in combination with one or more agents useful in the prevention or treatment of HIV. Examples of such agents include:

Nucleotide reverse transcriptase inhibitors such as zidovudine, didanosine, lamivudine, zalcitabine, abacavir, stavidine, adefovir, adefovir dipivoxil, fozivudine, todoxil, and similar agents;

Non-nucleotide reverse transcriptase inhibitors (including an agent having anti-oxidation activity such as immunocal, oltipraz, etc.) such as nevirapine, delavirdine, efavirenz, loviride, immunocal, oltipraz, and similar agents;

Protease inhibitors such as saquinavir, ritonavir, indinavir, nelfinavir, amprenavir, palinavir, lasinavir, and similar agents;

Entry inhibitors such as T-20, T-1249, PRO-542, PRO-140, TNX-355, BMS-806, 5-Helix and similar agents;

Integrase inhibitors such as L-870,180 and similar agents;

Budding inhibitors such as PA-344 and PA-457, and similar agents; and

Other CXCR4 and/or CCR5 inhibitors such as Sch-C, Sch-D, TAK779, UK 427,857, TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

The scope of combinations of compounds of this invention with HIV agents is not limited to those mentioned above, but includes in principle any combination with any pharmaceutical composition useful for the treatment of HIV. As noted, in such combinations the compounds of the present invention and other HIV agents may be administered separately or in conjunction. In addition, one agent may be prior to, concurrent to, or subsequent to the administration of other agent(s).

It should be understood that in addition to the ingredients particularly mentioned above, the pharmaceutical compositions of this invention may include other agents conventional in the art having regard to the type of pharmaceutical composition in question, for example, those suitable for oral administration may include such further agents as sweeteners, thickeners, and flavoring agents.

The compounds of the present invention may be prepared according to the following reaction schemes and examples, or modifications thereof using readily available starting materials, reagents and conventional synthesis procedures. In these reactions, it is also possible to make use of variants which are know to those of ordinary skill in the art.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

Those skilled in the art will recognize if a stereocenter exists in compounds of the present invention. Accordingly, the scope of the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### EXPERIMENTAL SECTION

### Abbreviations:

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| Hz (Hertz); | MHz (megahertz); |
| mol (moles); | mmol (millimoles); |
| RT (room temperature); | h (hours); |
| min (minutes); | TLC (thin layer chromatography); |
| mp (melting point); | RP (reverse phase); |
| Tᵣ (retention time); | TFA (trifluoroacetic acid); |
| TEA (triethylamine); | THF (tetrahydrofuran); |
| TFAA (trifluoroacetic anhydride); | CD₃OD (deuterated methanol); |
| CDCl₃ (deuterated chloroform); | DMSO (dimethylsulfoxide); |
| SiO₂ (silica); | atm (atmosphere); |
| EtOAc (ethyl acetate); | CHCl₃ (chloroform); |
| HCl (hydrochloric acid); | Ac (acetyl); |
| DMF (N,N-dimethylformamide); | Me (methyl); |
| Cs₂CO₃ (cesium carbonate); | EtOH (ethanol); |
| Et (ethyl); | tBu (tert-butyl); |
| MeOH (methanol) | p-TsOH (p-toluenesulfonic acid); |
| MP-TsOH (polystyrene resin bound | |
| equivalent of p-TsOH | |
| from Argonaut Technologies). | |

Unless otherwise indicated, all temperatures are expressed in ° C. (degrees Centigrade). All reactions conducted at room temperature unless otherwise noted.

¹H-NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on Micromass Platform or ZMD mass spectrometers from Micromass Ltd., Altricham, UK, using either Atmospheric Chemical Ionization (APCI) or Electrospray Ionization (ESI).

Analytical thin layer chromatography was used to verify the purity of intermediate(s) which could not be isolated or which were too unstable for full characterization as well as to follow the progress of reaction(s).

The absolute configuration of compounds was assigned by Ab Initio Vibrational Circular Dichroism (VCD) Spectroscopy. The experimental VCD spectra were acquired in CDCl₃ using a Bomem Chiral RTM VCD spectrometer operating between 2000 and 800 cm⁻¹. The Gaussian 98 Suite of computational programs was used to calculate model VCD spectrums. The stereochemical assignments were made by comparing this experimental spectrum to the VCD spectrum calculated for a model structure with (R)- or (S)-configuration. Incorporated by reference with regard to such spectroscopy are: J. R. Chesseman, M. J. Frisch, F. J. Devlin and P. J. Stephens, Chem. Phys. Lett. 252 (1996) 211; P. J. Stephens and F. J. Devlin, Chirality 12 (2000) 172; and Gaussian 98, Revision A. 11.4, M. J. Frisch et al., Gaussian, Inc., Pittsburgh Pa., 2002.

Compounds of formula (I) where all variables are as defined herein, and specifically wherein t is 1, each R is H, and all other variables are as defined above, can be prepared according to Scheme 1. Compounds of formula (I) wherein t is 0 or 2 and R is other than H can be made in a similar fashion as would be evident to one of skill in the art.

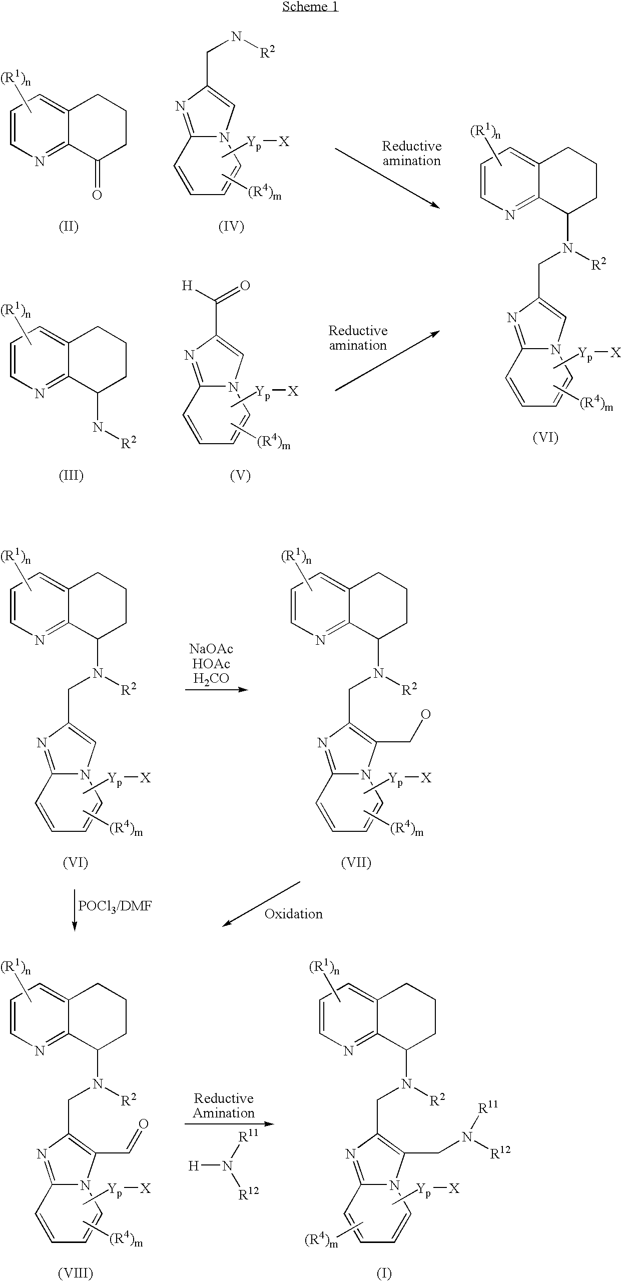

Generally, the process for preparing the compounds of formula (I) wherein t is 1, each R is H and all other variables are as defined herein above in connection with formula (I) comprises the steps of:

(a) preparing a compound of formula (VI) from a compound of formula (II) or (III) and a compound of formula (IV) or (V), respectively, by reductive amination.
(b) preparing a compound of formula (VIII) from a compound of formula (VI) via either of two methods:
1) formylation of a compound of formula (VI) using Vilsmeier Haack type conditions to give a compound of formula (VIII)
2) hydroxymethylation of a compound of formula (VI) to give a hydroxymethyl compound of formula (VI) followed by oxidation to give a compound of formula (VIII); and
   (c) preparing a compound of formula (I) from a compound of formula (VIII) by reductive amination;

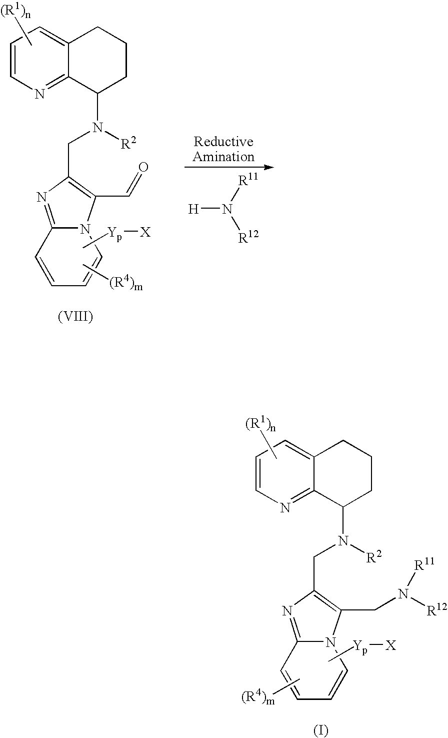

More specifically compounds of formula (I) can be prepared from compounds of formula (VIII) by reductive amination. The reductive amination can be carried out by treating the compound of formula (VIII) with an amine (HNR¹¹R¹²) in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like.

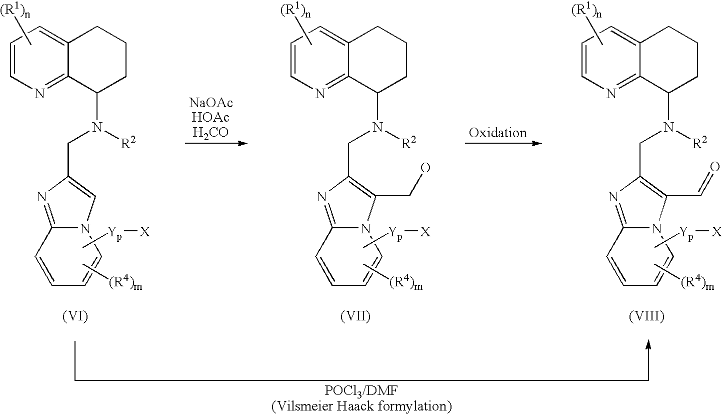

More specifically, compounds of formula (VIII) can be prepared from compounds of formula (VI) by either of two methods:
1) formylation of a compound of formula (VI) using Vilsmeier Haack type conditions to give a compound of formula (VIII) or
2) hydroxymethylation of a compound of formula (VI) to give a hydroxymethyl compound of formula (VII) followed by oxidation to give a compound of formula (VIII).

More specifically compounds of formula (VII) can be obtained by treatment of compound of formula (VI) with formaldehyde in the presence of acid, optionally in the presence of a solvent. The solvent can be acetic acid or an inert solvent such as water and the like. Optionally the reaction can be conducted at room temperature or with heating to 100° C. The reaction conditions are related to conditions described in the literature for hydroxymethylation of other imidazopyridines (e.g. Bioorganic and Medicinal Chemistry 2002,10, 941-946; J. Med. Chem. 1998, 41, 5108-5112 incorporated herein by reference with regard to such synthesis).

A compound of formula (VIII) can be obtained by oxidation of a compound of formula (VII). The oxidation is typically carried out in an inert solvent using a suitable oxidant. Suitable solvents include dichloromethane, chloroform, tetrahydrofuran and the like. Suitable oxidants include Dess Martin periodinane oxidation, preferentially using a Dess Marin periodinane on a solid support. The reaction can be carried out at room temperature or optionally with heating.

Alternatively, a compound of formula (VIII) can be prepared by formylation of compound of formula (VI) using Vilsmeier Haack formylation conditions (e.g. POCl3 and DMF) or other formylation conditions well know to those skilled in the art of organic chemistry.

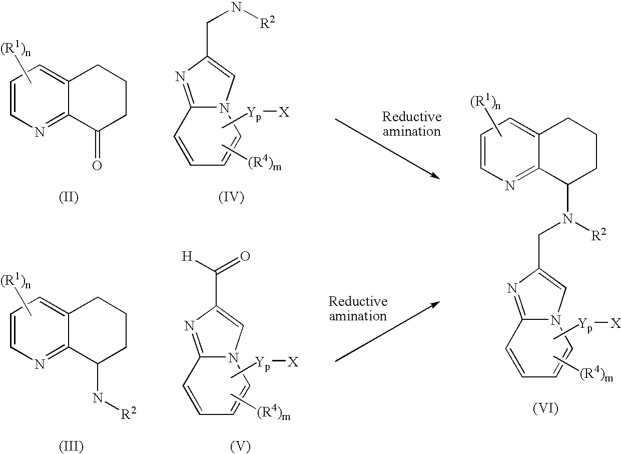

Compounds of formula (VI) can be prepared by reacting a compound of formula (II) with a compound (IV) or alternatively reacting a compound of formula. (III) with a compound of formula (V) under reductive conditions. The reductive amination can be carried out in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like.

Compounds of formula (II) can be prepared as described in the literature (J. Org. Chem., 2002, 67, 2197-2205, herein incorporated by reference with regard to such synthesis). Compounds of formula (III) can be prepared by reductive amination of compounds of formula (II) using processes well known to those skilled in the art of organic synthesis. Compounds of formula (V) can be prepared by methods similar to those described in the literature (J. Heterocyclic Chemistry, 1992, 29, 691-697, incorporated by reference with regard to such synthesis). Compounds of formula (IV) can be prepared from compounds of formula (V) via reductive amination using processes known to those skilled in the art.

Compounds of formula (I) wherein t is 1, each R is H, and all other variables are as defined herein, can be prepared according to Scheme 2. Compounds of formula (I) wherein t is 0 or 2 and R is other than H can be prepared in a similar fashion.

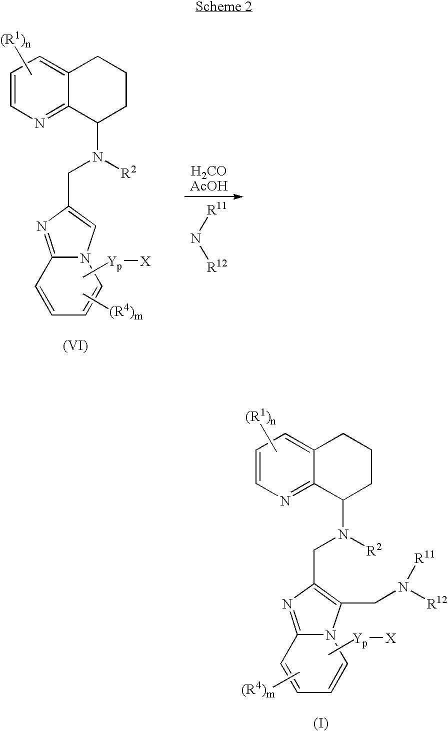

Compound of formula (I) can also be prepared by reacting a compound of formula (VI) with an amine and formaldehyde (Mannich reaction) using conditions well know to those skilled in the art of organic chemistry.

As is evident to one skilled in the art when at least one R¹¹ or R¹² is H a compound of formula I can be transformed into, for example, acylated, sulfonylated or urea compounds of formula I using conditions well know to those skilled in the art, e.g. via acylation, sulfonylation.

### EXAMPLES

### Example 1

### 6-Fluoro-2-pyridinamine (Intermediate)

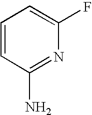

A solution of 2,6-difluoropyridine (50 g, 434 mmol) in ammonium hydroxide (200 mL, 28.0-30.0%) was heated at 105° C. in a steel bomb for 15 hours. The reaction was cooled in an ice bath and the precipitate filtered, rinsed with cold water, and dried to yield 6-fluoro-2-pyridinamine (45.8 g, 94% yield) as a white solid. ¹H-NMR (CDCl₃): δ 7.53 (m, 1H), 6.36 (dd, 1H), 6.26 (dd, 1H), 4.56 (s, 2H).

### Example 2

### 2-(Dichloromethyl)-5-fluoroimidazo[1,2-a]pyridine (Intermediate)

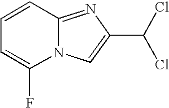

A solution of 6-fluoro-2-pyridinamine (67 g, 0.60 mol) in ethylene glycol dimethyl ether (570 mL) was treated with trichloroacetone (190 mL, 1.80 mol) and heated at 85° C. for 15 hours. The reaction was cooled in an ice bath and the precipitate filtered, rinsed with hexanes, and dried to yield 2-(dichloromethyl)-5-fluoroimidazo[1,2-a]pyridine (85g, 65% yield) as an olive green solid. ¹H-NMR (CDCl₃): δ 8.18 (s, 1H), 7.60 (s, 1H), 7.54-7.46 (m, 2H), 6.93 (m, 1H).

### Example 3

### 5-Fluoroimidazo[1,2-a]pyridine-2-carbaldehyde (Intermediate)

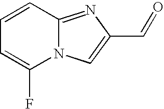

A solution of 2-(dichloromethyl)-5-fluoroimidazo[1,2-a]pyridine (103 g, 470 mmol) in ethanol (300 mL) and water (600 mL) was treated with sodium acetate (96 g, 1.17 mol) and heated at 60° C. for 2 hours. The reaction was cooled, filtered though celite, and concentrated in vacuo to remove the ethanol. The aqueous was extracted twice with chloroform and the organics were combined, washed with water and brine, dried over sodium sulfate, and concentrated. The residue was filtered through a pad of silica, rinsed with dichloromethane and ethyl acetate, concentrated, triturated with hexanes, filtered, and dried to yield 5-fluoroimidazo[1,2-a]pyridine-2-carbaldehyde (40 g, 52% yield) as a tan solid. ¹H-NMR (CDCl₃): δ 10.17 (s, 1H), 8.22 (s, 1H), 7.57 (d, 1H), 7.38-7.32 (m, 1H), 6.60 (m, 1H).

### Example 4

### (5-Fluoroimidazo[1,2-a]pyridin-2-yl)methanol (Intermediate)

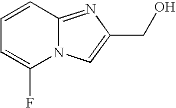

A solution of 5-fluoroimidazo[1,2-a]pyridine-2-carbaldehyde (80 g, 490 mmol) in methanol (1 L) at 0° C. was treated with sodium borohydride (24 g, 640 mmol) in portions. The reaction was slowly brought to room temperature, stirred for 2 hours, quenched with water, concentrated, dissolved in 3:1 dichloromethane to isopropyl alcohol, and washed with saturated aqueous sodium bicarbonate. The organic layer was separated and the aqueous extracted four times with 3:1 dichloromethane to isopropyl alcohol. The organic layers were combined, dried over sodium sulfate, concentrated, triturated with hexanes, and filtered to yield (5-fluoroimidazo[1,2-a]pyridin-2-yl)methanol (76g, 93% yield) as a brown solid. ¹H-NMR (CDCl₃): δ 7.59 (s, 1H), 7.38 (d, 1H), 7.21-7.15 (m, 1H), 6.43 (m, 1H), 4.85 (s, 2H), 4.45 (s, 1H).

### Example 5

### [5-(4-Methyl-1-piperazinyl)imidazo[1,2-a]pyridin-2-yl]methanol (Intermediate)

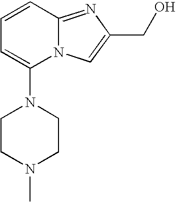

A solution of (5-fluoroimidazo[1,2-a]pyridin-2-yl)methanol (76 g, 460 mmol) in 1-methyl piperazine (150 mL) was heated at 70° C. for 15 hours. The reaction mixture was cooled, poured into 1.3 L brine, and extracted into 3:1 chloroform to isopropyl alcohol. The combined extracts were dried over sodium sulfate, concentrated in vacuo, azeotroped with hexanes, and triturated with diethyl ether to yield [5-(4-methyl-1-piperazinyl)imidazo[1,2-a]pyridin-2-yl]methanol (101g, 90% yield) as a tan solid. ¹H-NMR (CDCl₃): δ 7.51 (s, 1H), 7.33 (d, 1H), 7.21-7.17 (m, 1H), 6.31 (m, 1H), 4.87 (s, 2H), 3.17 (s, 4H), 2.68 (s, 4H), 2.42 (s, 3H).

### Example 6

### 5-(4-Methyl-1-piperazinyl)imidazo[1,2-a]pyridine-2-carbaldehyde (Intermediate)

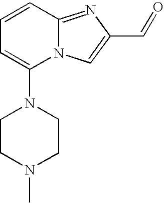

A solution of [5-(4-methyl-1-piperazinyl)imidazo[1,2-a]pyridin-2-yl]methanol (101 g, 410 mmol) in chloroform (1650 mL) was treated with manganese dioxide (360 g, 4100 mmol) and stirred at room temperature for 72 hours. The reaction mixture was filtered through celite, rinsed with chloroform, and concentrated to yield 5-(4-methyl-1-piperazinyl)imidazo[1,2-a]pyridine-2-carbaldehyde (82g, 82% yield) as gold solid. ¹H-NMR (CDCl₃): δ 10.17 (s, 1H), 8.15 (s, 1H), 7.44 (d, 1H), 7.31-7.27 (m, 1H), 6.40 (m, 1H), 3.16 (s, 4H), 2.68 (s, 4H), 2.42 (s, 3H).

### Example 7

### (8S)—N-[(1S)-1-[4-(Methyloxy)phenyl]ethyl]-5,6,7,8-tetrahydro-8-quinolinamine (Intermediate)

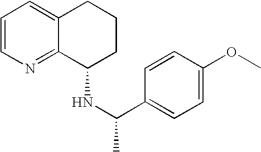

A solution of (S)-(−)-1-(4-methoxyphenyl)ethylamine (25 g, 166 mmol) and 6,7-dihydro-8(5H)-quinolinone (24 g, 166 mmol) in dichloromethane was treated with glacial acetic acid (14 mL, 249 mmol) and sodium triacetoxyborohydride (53 g, 249 mmol). The reaction mixture was stirred at room temperature for 15 hours and treated with sodium carbonate (106 g, 996 mmol) and stirred for 30 minutes. The mixture was diluted with dichloromethane, the organic layer separated, and the aqueous extracted with more dichloromethane. The organic layers were combined, dried over magnesium sulfate, concentrated, and purified by column chromatography (0-3% 2 M ammonia in methanol/dichloromethane) to give a yellow oil which was crystallized from hexanes to yield (8S)—N-{(1S)-1-[4-(methyloxy)phenyl]ethyl}-5,6,7,8-tetrahydro-8-quinolinamine (33g, 70% yield) as clear crystals. ¹H-NMR (CDCl₃): δ 8.40 (m, 1H), 7.33 (m, 3H), 7.04 (m, 1H), 6.84 (d, 2H), 4.02 (m, 1H), 3.83-3.78 (m, 4H), 2.73-2.62 (m, 2H), 1.82 (m, 1H), 1.72 (m, 1H), 1.57 (m, 2H), 1.43 (d, 3H).

### Example 8

### (8S)—N-{(1S)-1-[4-(Methyloxy)phenyl]ethyl}-N-{[5-(4-methyl-1-piperazinyl)imidazo[1,2-a]pyridin-2-yl]methyl}-5,6,7,8-tetrahydro-8-quinolinamine (Intermediate)

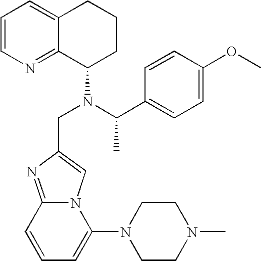

A solution of 5-(4-methyl-1-piperazinyl)imidazo[1,2-a]pyridine-2-carbaldehyde (2.83 g, 11.6 mmol) and (8S)—N-{(1S)-1-[4-(methyloxy)phenyl]ethyl}-5,6,7,8-tetrahydro-8-quinolinamine (3.27 g, 11.6 mmol) in dichloroethane (40 mL) was treated with glacial acetic acid (1.0 mL, 17.4 mmol) and sodium triacetoxyborohydride (3.68 g, 17.4 mmol, added in portions) and stirred at room temperature for 15 hours. The reaction mixture was diluted with dichloromethane, washed with saturated aqueous sodium bicarbonate, separated, and extracted with additional dichloromethane. The organic layers were combined, washed with brine, dried over sodium sulfate, concentrated, and purified by flash chromatography (0-4% ammonium hydroxide in acetonitrile). The residue was dissolved in dichloromethane and stirred with 2 M ammonia in methanol to yield (8S)—N-{(1S)-1-[4-(methyloxy)phenyl]ethyl}-N-{[5-(4-methyl-1-piperazinyl)imidazo[1,2-a]pyridin-2-yl]methyl}5,6,7,8-tetrahydro-8-quinolinamine (5.13 g, 87% yield) as pale yellow foam. ¹H-NMR (CDCl₃): δ 8.48 (m, 1H), 7.78 (s, 1H), 7.59 (d, 2H), 7.21 (m, 2H), 7.08 (m, 1H), 6.97 (m, 1H), 6.83 (d, 2H), 6.21 (d, 1H), 4.83 (m, 1H), 4.06 (s, 1H), 4.00-3.81 (m, 2H), 3.77 (s, 3H), 3.16 (m, 4H), 2.74 (m, 4H), 2.67-2.53 (m, 2H), 2.47 (s, 3H), 2.06 (m, 1H), 1.86 (m, 2H), 1.53 (m, 1H), 1.34 (d, 3H); MS m/z 511 (M+1)⁺.

### Example 9

### (8S)—N-Methyl-N-{[5-(4-methyl-1-piperazinyl)imidazo[1,2-a]pyridin-2-yl]methyl}-5,6,7,8-tetrahydro-8-quinolinamine (Intermediate)

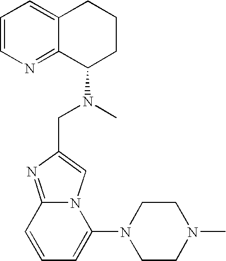

A solution of (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine (569 mg, 1.11 mmol) in dichloromethane (11.1 mL) was treated with trifluoroacetic acid (1.11 mL) and stirred at room temperature for 4 hours. The reaction was concentrated, diluted with dichloromethane, and washed with saturated aqueous sodium bicarbonate. The organic layer was separated and the aqueous extracted with dichloromethane. The organic layers were combined, dried over magnesium sulfate, filtered, and concentrated to yield (8S)—N-{[5-(4-methyl-1-piperazinyl)imidazo[1,2-a]pyridin-2-yl]methyl}-5,6,7,8-tetrahydro-8-quinolinamine as a yellow residue. ¹H-NMR (CDCl₃): δ 8.41 (d, 1H), 7.65 (s, 1H), 7.39 (d, 1H), 7.31 (m, 1H), 7.16 (m, 1H), 7.09 (m, 1H), 6.27 (dd, 1H), 4.31-4.17 (m, 2H), 4.05 (m, 1H), 3.15 (m, 4H), 2.88-2.78 (m, 2H), 2.67 (m, 4H), 2.41 (s, 3H), 2.29 (m, 1H), 2.08 (m, 1H), 1.96 (m, 1H), 1.77 (m, 1H). This residue was dissolved in dichloroethane (10 mL) and treated with formaldehyde (166 μL, 2.22 mmol, 37 wt. % solution in water), glacial acetic acid (96 μL, 1.67 mmol), sodium triacetoxyborohydride (353 mg, 1.67 mmol) and stirred at room temperature for 15 hours. The reaction was diluted with dichloromethane and washed with saturated aqueous sodium bicarbonate. The organic layer was separated and the aqueous extracted with dichloromethane. The organic layers were combined, dried over magnesium sulfate, filtered, concentrated, and purified by flash chromatography (0-10% ammonium hydroxide in acetonitrile) to give 2.76 g (64% yield, 2 steps) (8S)—N-methyl-N{[5-(4-methyl-1-piperazinyl)imidazo[1,2-a]pyridin-2-yl]methyl}-5,6,7,8-tetrahydro-8-quinolinamine as a pale yellow oil. ¹H-NMR (CDCl₃): δ 8.52 (d, 1H), 7.70 (s, 1H), 7.34 (d, 1H), 7.28 (d, 1H), 7.10 (m, 1H), 7.06 (m, 1H), 6.23 (dd, 1H), 4.12 (m, 1H), 3.96 (s, 2H), 3.14 (m, 4H), 2.86-2.78 (m, 2H), 2.71-2.65 (m, 4H), 2.40 (s, 6H), 2.16 (m, 1H), 2.06-1.97 (m, 2H), 1.68 (m, 1H); MS m/z 391 (M+1)⁺.

### Example 10

### (8S)—N-{[3-[(Dimethylamino)methyl]-5-(4-methyl-1-piperazinyl)imidazo[1,2-a]pyridin-2-yl]methyl}-N-methyl-5,6,7,8-tetrahydro-8-quinolinamine

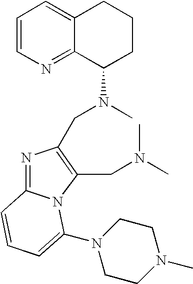

A solution of (8S)—N-methyl-N-{[5-(4-methyl-1-piperazinyl)imidazo[1,2-a]pyridin-2-yl]methyl}-5,6,7,8-tetrahydro-8-quinolinamine (100 mg, 0.25 mmol) in formaldehyde (192 μL) and glacial acetic acid (100 μL) was treated with dimethylamine (1.3 mL, 2.5 mmol, 2 M in tetrahydrofuran) and heated at 50° C. for 15 hours. The reaction mixture was diluted with dichloromethane and washed with saturated aqueous sodium carbonate. The organic layer was separated, the solvent removed, and the residue purified by flash chromatography (0-10% ammonium hydroxide in acetonitrile) to give (8S)—N-{[3-[(dimethylamino)methyl]-5-(4-methyl-1-piperazinyl)imidazo[1,2-a]pyridin-2-yl]methyl}-N-methyl-5,6,7,8-tetrahydro-8-quinolinamine (87 mg, 76% yield) as a yellow oil. ¹H NMR (400 MHz, CDCl₃) δ 8.57 (d, J=4.2 Hz, 1H), 7.35 (m, 1H), 7.31 (m, 1H), 7.08-7.03 (m, 2H), 6.42 (d, J=7.1 Hz, 1H), 4.08-4.02 (m, 2H), 3.92 (m, 1H), 3.79-3.70 (m, 2H), 3.21-3.15 (m, 2H), 3.04 (m, 1H), 2.89-2.76 (m, 3H), 2.72-2.59 (m, 2H), 2.52-2.42 (m, 2H), 2.38 (s, 6H), 2.12-2.01 (m, 3H), 1.93 (s, 6H), 1.69 (m, 1H); MS m/z 470 (M+Na)⁺.

### Example 11

### (8S)—N-{[3-[(Diethylamino)methyl]-5-(4-methyl-1-piperazinyl)imidazo[1,2-a]pyridin-2-yl]methyl}-N-methyl-5,6,7,8-tetrahydro-8-quinolinamine

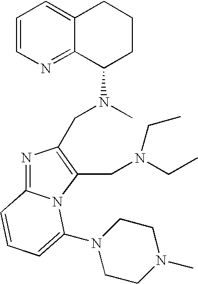

(8S)—N-{[3-[(Diethylamino)methyl]-5-(4-methyl-1-piperazinyl)imidazo[1,2-a]pyridin-2-yl]methyl}-N-methyl-5,6,7,8-tetrahydro-8-quinolinamine was prepared from (8S)—N-methyl-N{[5-(4-methyl-1-piperazinyl)imidazo[1,2-a]pyridin-2-yl]methyl}-5,6,7,8-tetrahydro-8-quinolinamine and diethylamine in a similar manner as described herein above to give a yellow oil (25% yield). ¹H NMR (400 MHz, CDCl₃) δ 8.50 (d, J=4.0 Hz, 1H), 7.35-7.31 (m, 2H), 7.07-7.02 (m, 2H), 6.41 (d, J=7.3 Hz, 1H), 4.22-4.07 (m, 3H), 3.88 (s, 2H), 3.19-3.13 (m, 3H), 2.99 (m, 1H), 2.87-2.77 (m, 2H), 2.72-2.65 (m, 2H), 2.48-2.40 (m, 4H), 2.37 (s, 3H), 2.32 (s, 3H), 2.13-2.01 (m, 3H), 1.70 (m, 1H), 0.78 (s, 6H); MS m/z 498 (M+Na)⁺.

### Example 12

### (8S)—N-Methyl-N-{[5-(4-methyl-1-piperazinyl)-3-(1-pyrrolidinylmethyl)imidazo[1,2-a]pyridin-2-yl]methyl}-5,6,7,8-tetrahydro-8-quinolinamine

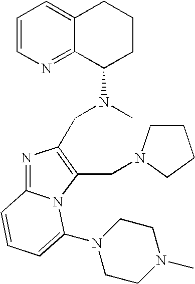

(8S)—N-Methyl-N-{[5-(4-methyl-1-piperazinyl)-3-(1-pyrrolidinylmethyl)imidazol-[1,2-a]pyridin-2-yl]methyl}-5,6,7,8-tetrahydro-8-quinolinamine was prepared from (8S)—N-methyl-N-{[5-(4-methyl-1-piperazinyl)imidazo[1,2-a]pyridin-2-yl]methyl}-5,6,7,8-tetrahydro-8-quinolinamine and pyrrolidine in a similar manner as described herein above to give a yellow oil (51% yield). ¹H NMR (400 MHz, CDCl₃) δ 8.50 (d, J=4.1 Hz, 1H), 7.33-7.27 (m, 2H), 7.05-7.00 (m, 2H), 6.39 (d, J=7.1 Hz, 1H), 4.37-4.27 (m, 2H), 4.03 (t, J=7.1 Hz, 1H), 3.81-3.74 (m, 2H), 3.16-3.11 (m, 2H), 2.98 (m, 1H), 2.85-2.74 (m, 3H), 2.65 (m, 1H), 2.47-2.37 (m, 6H), 2.34 (s, 3H), 2.29 (s, 3H), 2.17 (s, 2H), 2.07-1.97 (m, 3H), 1.66 (m, 1H), 1.50 (s, 3H); MS m/z 496 (M+Na)⁺.

### Example 13

### 2,2′-({[5-(4-Methyl-1-piperazinyl)-2-({methyl[(8S)-5,6,7,8-tetrahydro-8-quinolinyl]amino}methyl)imidazo[1,2-a]pyridin-3-yl]methyl}imino)diethanol

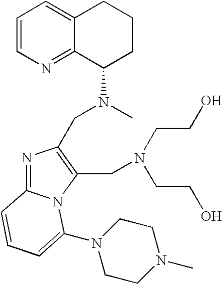

2,2′-({[5-(4-Methyl-1-piperazinyl)-2-({methyl[(8S)-5,6,7,8-tetrahydro-8-quinolinyl]amino}methyl)imidazo[1,2-a]pyridin-3-yl]methyl}imino)diethanol was prepared from (8S)—N-methyl-N-{[5-(4-methyl-1-piperazinyl)imidazo[1,2-a]pyridin-2-yl]methyl}-5,6,7,8-tetrahydro-8-quinolinamine and diethanolamine in a similar manner as described herein above to give a clear oil (18% yield). ¹H NMR (400 MHz, CDCl₃) δ 8.41 (d, J=4.6 Hz, 1H), 7.38-7.33 (m, 2H), 7.10 (t, J=7.9 Hz, 1H), 7.05-7.02 (m, 1H), 6.49 (d, J=7.1 Hz, 1H), 4.50 (m, 2H), 4.20 (m, 1H), 4.04-3.95 (m, 2H), 3.45 (m, 4H), 3.16-3.11 (m, 2H), 2.95-2.85 (m, 4H), 2.80-2.75 (m, 1H), 2.71-2.67 (m, 5H), 2.53-2.46 (m, 2H), 2.41 (s, 3H), 2.30 (m, 1H), 2.15 (s, 3H), 2.09-2.02 (m, 2H), 1.71 (m, 1H); MS m/z 530 (M+Na)⁺.

### Example 14

### 2-(Ethyl{[5-(4-methyl-1-piperazinyl)-2-({methyl[(8S)-5,6,7,8-tetrahydro-8-quinolinyl]amino}methyl)imidazo[1,2-a]pyridin-3-yl]methyl}amino)ethanol

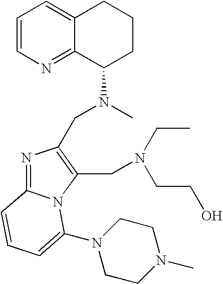

2-(Ethyl{[5-(4-methyl-1-piperazinyl)-2-({methyl[(8S)-5,6,7,8-tetrahydro-8-quinolinyl]amino}methyl)imidazo[1,2-a]pyridin-3-yl]methyl}amino)ethanol was prepared from (8S)—N-methyl-N-{[5-(4-methyl-1-piperazinyl)imidazo[1,2-a]pyridin-2-yl]methyl}-5,6,7,8-tetrahydro-8-quinolinamine and 2-ethylaminoethanol in a similar manner as described herein above to give an off-white solid (71% yield). ¹H NMR MHz, CDCl₃) δ 8.47 (m, 1H), 7.34-7.28 (m, 2H), 7.07-7.00 (m, 2H), 6.40 (d, J=7.1 Hz, 1H), 4.31-4.24 (m, 2H), 4.07 (m, 1H), 3.89 (s, 2H), 3.64 (s, 1H), 3.19-3.09 (m, 4H), 2.96 (m, 1H), 2.87-2.75 (m, 4H), 2.73-2.64 (m, 3H), 2.50-2.39 (m, 7H), 2.36 (s, 3H), 2.33 (s, 3H), 2.11 (m, 1H), 2.03-1.97 (m, 2H), 1.67 (m, 1H); MS m/z 514 (M+Na)⁺.

### Example 15

### 1-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-3-pyrrolidinol

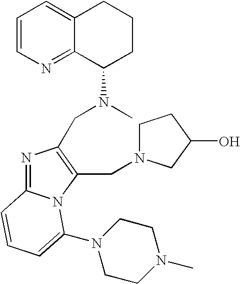

1-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-3-pyrrolidinol was prepared from (8S)—N-methyl-N-{[5-(4-methyl-1-piperazinyl)imidazo[1,2-a]pyridin-2-yl]methyl}-5,6,7,8-tetrahydro-8-quinolinamine and 3-pyrrolidinol in a similar manner as described herein above to give a white solid (70% yield). ¹H NMR (400 MHz, CDCl₃) δ 8.55 (m, 1H), 7.38 (d, J=7.7 Hz, 1H), 7.33 (d, J=8.7 Hz, 1H), 7.10-7.06 (m, 2H), 6.44 (d, J=7.2 Hz, 1H), 4.37 (m, 1H), 4.28 (m, 1H), 4.11-4.04 (m, 2H), 3.78 (s, 2H), 3.21-3.14 (m, 2H), 3.11-2.99 (m, 2H), 2.90-2.44 (m, 6H), 2.39 (s, 3H), 2.35 (d, J=5.2 Hz, 3H), 2.24-2.03 (m, 4H), 1.96-1.85 (m, 2H), 1.71 (m, 1H), 1.52 (m, 1H); MS m/z 512 (M+Na)⁺.

### Example 16

### (8S)—N-Methyl-N-{[5-(4-methyl-1-piperazinyl)-3-(4-morpholinylmethyl)imidazo[1,2-a]pyridin-2-yl]methyl}-5,6,7,8-tetrahydro-8-quinolinamine

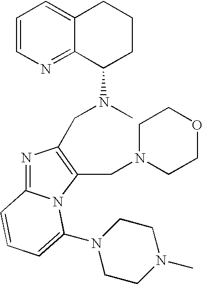

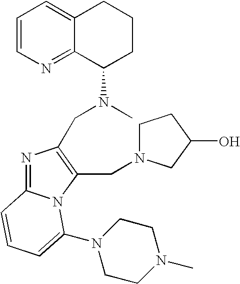

1-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-3-pyrrolidinol was prepared from (8S)—N-methyl-N-{[5-(4-methyl-1-piperazinyl)imidazo[1,2-a]pyridin-2-yl]methyl}-5,6,7,8-tetrahydro-8-quinolinamine and 3-pyrrolidinol in a similar manner as described herein above to give a white solid (70% yield). ¹H NMR (400 MHz, CDCl₃) δ 8.55 (m, 1H), 7.38 (d, J=7.7 Hz, 1H), 7.33 (d, J=8.7 Hz, 1H), 7.10-7.06 (m, 2H), 6.44 (d, J=7.2 Hz, 1H), 4.37 (m, 1H), 4.28 (m, 1H), 4.11-4.04 (m, 2H), 3.78 (s, 2H), 3.21-3.14 (m, 2H), 3.11-2.99 (m, 2H), 2.90-2.44 (m, 6H), 2.39 (s, 3H), 2.35 (d, J=5.2 Hz, 3H), 2.24-2.03 (m, 4H), 1.96-1.85 (m, 2H), 1.71 (m, 1H), 1.52 (m, 1H); MS m/z 512 (M+Na)⁺.

### Example 16

### (8S)—N-Methyl-N-{[5-(4-methyl-1-piperazinyl)-3-(4-morpholinylmethyl)imidazo[2-a]pyridin-2-yl]methyl}-5,6,7,8-tetrahydro-8-quinolinamine

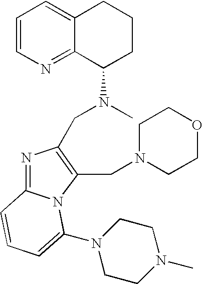

(8S)—N-Methyl-N-{[5-(4-methyl-1-piperazinyl)-3-(4-morpholinylmethyl)imidazo[1,2-a]pyridin-2-yl]methyl}-5,6,7,8-tetrahydro-8-quinolinamine was prepared from (8S)—N-methyl-N-{[5-(4-methyl-1-piperazinyl)imidazo[1,2-a]pyridin-2-yl]methyl}-5,6,7,8-tetrahydro-8-quinolinamine and morpholine in a similar manner as described herein above to give a clear oil (75% yield). ¹H NMR (400 MHz, CDCl₃) δ 8.57 (d, J=4.4 Hz, 1H), 7.37 (d, J=7.5 Hz, 1H), 7.33 (d, J=8.7 Hz, 1H), 7.09-7.05 (m, 2H), 6.47 (d, J=7.1 Hz, 1H), 4.16-3.99 (m, 3H), 3.76 (m, 2H), 3.43 (s, 4H), 3.17-3.12 (m, 2H), 3.04 (m, 1H), 2.88-2.77 (m, 3H), 2.74-2.67 (m, 2H), 2.41 (m, 2H), 2.39 (s, 3H), 2.36 (s, 3H), 2.19-2.03 (m, 7H), 1.70 (m, 1H); MS m/z 512 (M+Na)⁺.

### Example 17

### [5-(4-Methyl-1-piperazinyl)-2-({methyl[(8S)-5,6,7,8-tetrahydro-8-quinolinyl]amino}methyl)imidazo[1,2-a]pyridin-3-yl]methanol (Intermediate)

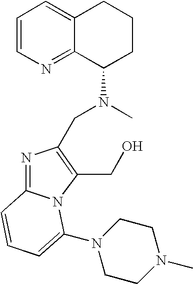

A solution of (8S)—N-methyl-N-{[5-(4-methyl-1-piperazinyl)imidazo[1,2-a]pyridin-2-yl]methyl}-5,6,7,8-tetrahydro-8-quinolinamine (2.9 g, 7.4 mmol) in formaldehyde (10 mL, 37 wt. % solution in water) and glacial acetic acid (2.5 mL) was heated at 50° C. for 15 hours. Reaction mixture was cooled, diluted with dichloromethane, and washed with saturated aqueous sodium carbonate. The organic layer was isolated and the aqueous washed three times with dichloromethane/isopropyl alcohol. The organic layers were combined, dried with magnesium sulfate, filtered, and concentrated. The residue was purified by flash chromatography (0-10% ammonium hydroxide in acetonitrile) to give 2.1 g (68% yield) [5-(4-methyl-1-piperazinyl)-2-({methyl[(8S)-5,6,7,8-tetrahydro-8-quinolinyl]amino}methyl)imidazo[1,2-a]pyridin-3-yl]methanol as a white solid. The solid was recrystallized from dichloromethane and hexanes. ¹H-NMR (CDCl₃): δ 8.42 (d, 1H), 7.31 (m, 2H), 7.06 (m, 1H), 7.01 (m, 1-H), 6.75 (s, 1H), 6.39 (dd, 1H), 5.29 (m, 2H), 4.01 (m, 3H), 3.52 (m, 1H), 3.38 (m, 1H), 2.90 (m, 4H), 2.78 (m, 1H), 2.67 (m, 1H), 2.52 (m, 2H), 2.40 (s, 3H), 2.21 (m, 1H), 2.13 (s, 3H), 1.96 (m, 2H), 1.68 (m, 1H); MS m/z 443 (M+Na)⁺.

### Example 18

### 5-(4-Methyl-1-piperazinyl)-2-({methyl[(8S)-5,6,7,8-tetrahydro-8-quinolinyl]amino}methyl)imidazo[1,2-a]pyridine-3-carbaldehyde (Intermediate)

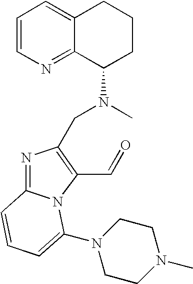

A solution of [5-(4-methyl-1-piperazinyl)-2-({methyl[(8S)-5,6,7,8-tetrahydro-8-quinolinyl]amino}methyl)imidazo[1,2-a]pyridin-3-yl]methanol (572 mg, 1.36 mmol) in dichloromethane (7 mL) was treated with IBX polystyrene (2 g, 2.8 mmol), stirred at room temperature for 15 hours, treated with additional IBX polystyrene (3 g, 4.2 mmol), and stirred at room temperature 24 hours. The reaction mixture was filtered, rinsed with dichloromethane, dissolved in methanol, heated at 40° C. for 15 hours, filtered, concentrated, and purified by flash chromatography (0-10% ammonium hydroxide in acetonitrile) to give 330 mg (58% yield) of 5-(4-methyl-1-piperazinyl)-2-({methyl[(8S)-5,6,7,8-tetrahydro-8-quinolinyl]amino}methyl)imidazo[1,2-a]pyridine-3-carbaldehyde as an orange oil. ¹H-NMR (CDCl₃): δ 10.86 (s, 1H), 8.47 (d, 1H), 7.47 (d, 1H), 7.36 (m, 2H), 7.04 (m, 1H), 6.61 (dd, 1H), 4.29 (s, 2H), 4.23 (m, 1H), 3.35 (m, 2H), 2.94-2.81 (m, 4H), 2.71-2.67 (m, 2H), 2.50 (s, 3H), 2.40 (m, 2H), 2.37 (s, 3H), 2.19 (m, 1H), 2.08 (m, 2H), 1.71 (m, 1H).

### Example 19

### (8S)—N-Methyl-N-{[3-[(methylamino)methyl]-5-(4-methyl-1-piperazinyl)imidazo[1,2-a]pyridin-2-yl]methyl}-5,6,7,8-tetrahydro-8-quinolinamine

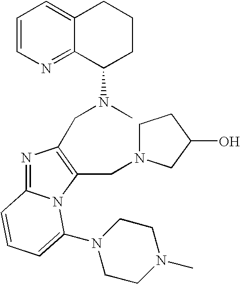

1-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-3-pyrrolidinol was prepared from (8S)—N-methyl-N-{[5-(4-methyl-1-piperazinyl)imidazo[1,2-a]pyridin-2-yl]methyl}-5,6,7,8-tetrahydro-8-quinolinamine and 3-pyrrolidinol in a similar manner as described herein above to give a white solid (70% yield). ¹H NMR (400 MHz, CDCl₃) δ 8.55 (m, 1H), 7.38 (d, J=7.7 Hz, 1H), 7.33 (d, J=8.7 Hz, 1H), 7.10-7.06 (m, 2H), 6.44 (d, J=7.2 Hz, 1H), 4.37 (m, 1H), 4.28 (m, 1H), 4.11-4.04 (m, 2H), 3.78 (s, 2H), 3.21-3.14 (m, 2H), 3.11-2.99 (m, 2H), 2.90-2.44 (m, 6H), 2.39 (s, 3H), 2.35 (d, J=5.2 Hz, 3H), 2.24-2.03 (m, 4H), 1.96-1.85 (m, 2H), 1.71 (m, 1H), 1.52 (m, 1H); MS m/z 512 (M+Na)⁺.

### Example 16

### (8S)—N-Methyl-N-{[5-(4-methyl-1-piperazinyl)-3-(4-morpholinylmethyl)imidazo[1,2-a]pyridin-2-yl]methyl}-5,6,7,8-tetrahydro-8-quinolinamine

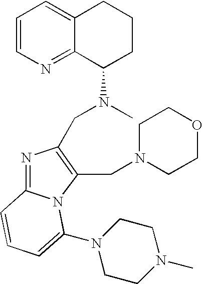

(8S)—N-Methyl-N-{[5-(4-methyl-1-piperazinyl)-3-(4-morpholinylmethyl)imidazo[1,2-a]pyridin-2-yl]methyl}-5,6,7,8-tetrahydro-8-quinolinamine was prepared from (8S)—N-methyl-N-{[5-(4-methyl-1-piperazinyl)imidazo[1,2-a]pyridin-2-yl]methyl}-5,6,7,8-tetrahydro-8-quinolinamine and morpholine in a similar manner as described herein above to give a clear oil (75% yield). ¹H NMR (400 MHz, CDCl₃) δ 8.57 (d, J=4.4 Hz, 1H), 7.37 (d, J=7.5 Hz, 1H), 7.33 (d, J=8.7 Hz, 1H), 7.09-7.05 (m, 2H), 6.47 (d, J=7.1 Hz, 1H), 4.16-3.99 (m, 3H), 3.76 (m, 2H), 3.43 (s, 4H), 3.17-3.12 (m, 2H), 3.04 (m, 1H), 2.88-2.77 (m, 3H), 2.74-2.67 (m, 2H), 2.41 (m, 2H), 2.39 (s, 3H), 2.36 (s, 3H), 2.19-2.03 (m, 7H), 1.70 (m, 1H); MS m/z 512 (M+Na)⁺.

### Example 17

### [5-(4-Methyl-1-piperazinyl)-2-({methyl[(8S)-5,6,7,8-tetrahydro-8-quinolinyl]amino}methyl)imidazo[1,2-a]pyridin-3-yl]methanol (Intermediate)

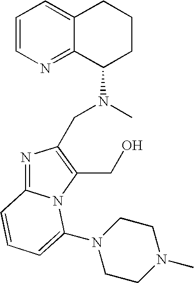

A solution of (8S)—N-methyl-N-{[5-(4-methyl-1-piperazinyl)imidazo[1,2-a]pyridin-2-yl]methyl}-5,6,7,8-tetrahydro-8-quinolinamine (2.9 g, 7.4 mmol) in formaldehyde (10 mL, 37 wt. % solution in water) and glacial acetic acid (2.5 mL) was heated at 50° C. for 15 hours. Reaction mixture was cooled, diluted with dichloromethane, and washed with saturated aqueous sodium carbonate. The organic layer was isolated and the aqueous washed three times with dichloromethane/isopropyl alcohol. The organic layers were combined, dried with magnesium sulfate, filtered, and concentrated. The residue was purified by flash chromatography (0-10% ammonium hydroxide in acetonitrile) to give 2.1 g (68% yield) [5-(4-methyl-1-piperazinyl)-2-({methyl[(8S)-5,6,7,8-tetrahydro-8-quinolinyl]amino}methyl)imidazo[1,2-a]pyridin-3-yl]methanol as a white solid. The solid was recrystallized from dichloromethane and hexanes. ¹H-NMR (CDCl₃): δ 8.42 (d, 1H), 7.31 (m, 2H), 7.06 (m, 1H), 7.01 (m, 1-H), 6.75 (s, 1H), 6.39 (dd, 1H), 5.29 (m, 2H), 4.01 (m, 3H), 3.52 (m, 1H), 3.38 (m, 1H), 2.90 (m, 4H), 2.78 (m, 1H), 2.67 (m, 1H), 2.52 (m, 2H), 2.40 (s, 3H), 2.21 (m, 1H), 2.13 (s, 3H), 1.96 (m, 2H), 1.68 (m, 1H); MS m/z 443 (M+Na)⁺.

### Example 18

### 5-(4-Methyl-1-piperazinyl)-2-({methyl[(8S)-5,6,7,8-tetrahydro-8-quinolinyl]amino}methyl)imidazo[1,2-a]pyridine-3-carbaldehyde (Intermediate)

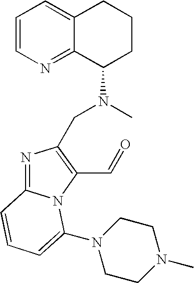

A solution of [5-(4-methyl-1-piperazinyl)-2-({methyl[(8S)-5,6,7,8-tetrahydro-8-quinolinyl]amino}methyl)imidazo[1,2-a]pyridin-3-yl]methanol (572 mg, 1.36 mmol) in dichloromethane (7 mL) was treated with IBX polystyrene (2 g, 2.8 mmol), stirred at room temperature for 15 hours, treated with additional IBX polystyrene (3 g, 4.2 mmol), and stirred at room temperature 24 hours. The reaction mixture was filtered, rinsed with dichloromethane, dissolved in methanol, heated at 40° C. for 15 hours, filtered, concentrated, and purified by flash chromatography (0-10% ammonium hydroxide in acetonitrile) to give 330 mg (58% yield) of 5-(4-methyl-1-piperazinyl)-2-({methyl[(8S)-5,6,7,8-tetrahydro-8-quinolinyl]amino}methyl)imidazo[1,2-a]pyridine-3-carbaldehyde as an orange oil. ¹H-NMR (CDCl₃): δ 10.86 (s, 1H), 8.47 (d, 1H), 7.47 (d, 1H), 7.36 (m, 2H), 7.04 (m, 1H), 6.61 (dd, 1H), 4.29 (s, 2H), 4.23 (m, 1H), 3.35 (m, 2H), 2.94-2.81 (m, 4H), 2.71-2.67 (m, 2H), 2.50 (s, 3H), 2.40 (m, 2H), 2.37 (s, 3H), 2.19 (m, 1H), 2.08 (m, 2H), 1.71 (m, 1H).

### Example 19

### (8S)—N-Methyl-N-{[3-[(methylamino)methyl]-5-(4-methyl-1-piperazinyl)imidazo[1,2-a]pyridin-2-yl]methyl}-5,6,7,8-tetrahydro-8-quinolinamine

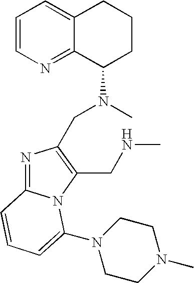

A solution of 5-(4-methyl-1-piperazinyl)-2-({methyl[(8S)-5,6,7,8-tetrahydro-8-quinolinyl]amino}methyl)imidazo[1,2-a]pyridine-3-carbaldehyde (50 mg, 0.12 mmol) in dichloroethane was treated with methylamine (90 μL, 0.18 mmol, 2 M in tetrahydrofuran), glacial acetic acid (10 μL, 0.18 mmol), sodium triacetoxyborohydride (38 mg, 0.18 mmol) and stirred at room temperature for 15 hours. The reaction was diluted with dichloromethane and washed with saturated aqueous sodium carbonate. The organic layer was separated, concentrated, and purified by flash chromatography (0-10% ammonium hydroxide in acetonitrile) to give 25 mg (48% yield) (8S)—N-Methyl-N-{[3-[(methylamino)methyl]-5-(4-methyl-1-piperazinyl)imidazo[1,2-a]pyridin-2-yl]methyl}-5,6,7,8-tetrahydro-8-quinolinamine as a pale orange oil. ¹H NMR (400 MHz, CDCl₃) δ 8.56 (d, J=4.4 Hz, 1H), 7.37 (s, 1H), 7.35 (m, 1H), 7.11-7.05 (m, 2H), 6.46 (d, J=7.1 Hz, 1H), 4.29 (s, 2H), 4.04 (m, 1H), 3.81 (d, J=3.7 Hz, 2H), 3.26-3.16 (m, 2H), 3.04 (m, 1H), 2.92-2.80 (m, 4H), 2.70 (m, 1H), 2.54-2.44 (m; 2H), 2.41 (s, 3H), 2.30 (s, 3H), 2.16 (s, 3H), 2.13 (m, 1H), 2.08-2.02 (m, 2H), 1.71 (m, 1H); MS m/z 456 (M+Na)⁺.

### Example 20

### (8S)—N-{[3-[(Ethylamino)methyl]-5-(4-methyl-1-piperazinyl)imidazo[1,2-a]pyridin-2-yl]methyl}-N-methyl-5,6,7,8-tetrahydro-8-quinolinamine

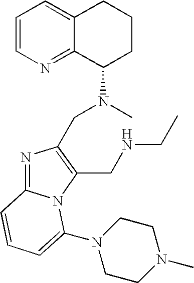

(8S)—N-{[3-[(Ethylamino)methyl]-5-(4-methyl-1-piperazinyl)imidazo[1,2-a]pyridin-2-yl]methyl}-N-methyl-5,6,7,8-tetrahydro-8-quinolinamine was prepared from 5-(4-methyl-1-piperazinyl)-2-({methyl[(8S)-5,6,7,8-tetrahydro-8-quinolinyl]amino}methyl)imidazo[1,2-a]pyridine-3-carbaldehyde and ethylamine in a similar manner as described in Example 19 to give a tan solid (54% yield). ¹H NMR (400 MHz, CDCl₃) δ 8.54 (m, 1H), 7.35-7.32 (m, 2H), 7.07-7.03 (m, 2H), 6.42 (d, J=7.1 Hz, 1H), 4.19 (s, 2H), 4.03 (m, 1H), 3.77 (s, 2H), 3.24 (d, J=10.6 Hz, 1H), 3.15-3.03 (m, 2H), 2.92-2.65 (m, 6H), 2.51-2.44 (m, 3H), 2.38 (s, 3H), 2.33-2.28 (m, 5H), 2.12-1.99 (m, 4H), 1.70 (m, 1H); MS m/z 447 (M⁺).

### Example 21

### (8S)—N-Methyl-N-({5-(4-methyl-1-piperazinyl)-3-[(4-methyl-1-piperazinyl)methyl]imidazo[1,2-a]pyridin-2-yl}methyl)-5,6,7,8-tetrahydro-8-quinolinamine

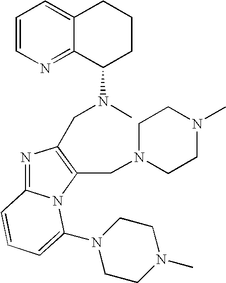

(8S)—N-Methyl-N-({5-(4-methyl-1-piperazinyl)-3-[(4-methyl-1-piperazinyl)methyl]imidazo[1,2-a]pyridin-2-yl}methyl)-5,6,7,8-tetrahydro-8-quinolinamine was prepared from (8S)—N-methyl-N-{[5-(4-methyl-1-piperazinyl)imidazo[1,2-a]pyridin-2-yl]methyl}-5,6,7,8-tetrahydro-8-quinolinamine and 1-methyl piperazine in a similar manner as described herein to give an orange oil (83% yield). ¹H NMR (400 MHz, CDCl₃) δ 8.55 (d, J=4.5 Hz, 1H), 7.36-7.33 (m, 2H), 7.09-7.05 (m, 2H), 6.46 (d, J=7.1 Hz, 1H), 4.19 (d, J=13.4 Hz, 1H), 4.06 (m, 1H), 4.01 (d, J=13.8 Hz, 1H), 3.80 (q, J=13.6 Hz, 2H), 3.17-3.14 (m, 2H), 3.01 (m, 1H), 2.88-2.66 (m, 8H), 2.53-2.34 (m, 10H), 2.26 (s, 3H), 2.23 (m, 1H), 2.15 (s, 3H), 2.11-2.01 (m, 2H), 1.71 (m, 1H); MS m/z 503 (M+1).

### Example 22

### (8S)—N-Methyl-N-{[5-(4-methyl-1-piperazinyl)-3-(1-piperidinylmethyl)imidazo[1,2-a]pyridin-2-yl]methyl}-5,6,7,8-tetrahydro-8-quinolinamine

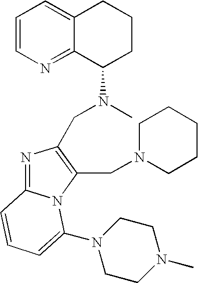

8S)—N-Methyl-N-{[5-(4-methyl-1-piperazinyl)-3-(1-piperidinylmethyl)imidazo[1,2-a]pyridin-2-yl]methyl}-5,6,7,8-tetrahydro-8-quinolinamine was prepared from (8S)—N-methyl-N-{[5-(4-methyl-1-piperazinyl)imidazo[1,2-a]pyridin-2-yl]methyl}-5,6,7,8-tetrahydro-8-quinolinamine and piperidine in a similar manner as described herein to give a pale yellow oil (82% yield). ¹H NMR (400 MHz, CDCl₃) δ 8.54 (d, J=4.5 Hz, 1H), 7.35-7.31 (m, 2H), 7.06-7.03 (m, 2H), 6.44 (d, J=7.2 Hz, 1H), 4.08 (d, J=13.5 Hz, 1H), 4.03 (m, 1H), 3.91 (d, J=13.6 Hz, 1H), 3.77 (d, J=4.4 Hz, 2H), 3.17-3.11 (m, 2H), 2.98 (m, 1H), 2.85-2.64 (m, 5H), 2.45-2.39 (m, 2H), 2.35 (s, 3H), 2.30 (s, 3H), 2.10-2.00 (m, 6H), 1.68 (m, 1H), 1.49 (m, 1H), 1.31-1.22 (m, 6H); MS m/z 488 (M+1).

### Example 23

### (8S)—N-Methyl-N-{[3-{[(1-methylethyl)amino]methyl}-5-(4-methyl-1-piperazinyl)imidazo[1,2-a]pyridin-2-yl]methyl}-5,6,7,8-tetrahydro-8-quinolinamine

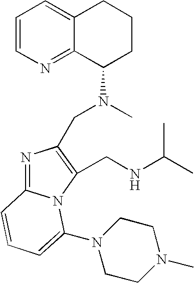

(8S)—N-Methyl-N-{[3-{[(1-methylethyl)amino]methyl}-5-(4-methyl-1-piperazinyl)imidazo[1,2-a]pyridin-2-yl]methyl}-5,6,7,8-tetrahydro-8-quinolinamine was prepared from 5-(4-methyl-1-piperazinyl)-2-({methyl[(8S)-5,6,7,8-tetrahydro-8-quinolinyl]amino}methyl)imidazo[1,2-a]pyridine-3-carbaldehyde and isopropylamine in a similar manner as described herein to give a tan solid (69% yield). ¹H NMR (400 MHz, CD₃OD) δ 8.47 (d, J=4.4 Hz, 1H), 7.53 (d, J=7.6 Hz, 1H), 7.34-7.32 (m, 2H), 7.20 (dd, J=7.7, 4.7 Hz, 1H), 6.77 (dd, J=6.2, 2.2 Hz, 1H), 4.88 (s, 2H), 4.54 (br, 1H), 4.43 (m, 1H), 3.98 (m, 1H), 3.85 (s, 2H), 3.32-3.25 (m, 2H), 3.02-2.85 (m, 4H), 2.80-2.73 (m, 2H), 2.55-2.48 (m, 2H), 2.43 (s, 3H), 2.27 (s, 3H), 2.19 (m, 1H), 2.12-2.05 (m, 2H), 1.74 (m, 1H), 1.10-1.04 (m, 6H); MS m/z 462 (M+1).

### Example 24

### (8S)—N-{[3-(Aminomethyl)-5-(4-methyl-1-piperazinyl)imidazo[1,2-a]pyridin-2-yl]methyl}-N-methyl-5,6,7,8-tetrahydro-8-quinolinamine

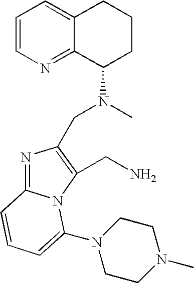

To a solution of 5-(4-methyl-1-piperazinyl)-2-({methyl[(8S)-5,6,7,8-tetrahydro-8-quinolinyl]amino}methyl)imidazo[1,2-a]pyridine-3-carbaldehyde (1.0 g, 2.39 mmol) and ammonium acetate (1.84 g, 23.9 mmol) in methanol (20 mL) was added sodium cyanoborohydride (751 mg, 12.0 mmol). After heating at 50° C. for 15 hours, the mixture was cooled, treated with saturated aqueous sodium carbonate, and concentrated. The residue was diluted with dichloromethane and washed with saturated aqueous sodium carbonate. The organics were separated and the aqueous extracted two times with dichloromethane/isopropyl alcohol. The organics were combined, concentrated, and purified by flash chromatography (0-10% ammonium hydroxide in acetonitrile) to give 0.49 g (49% yield) (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine as a tan solid. ¹H NMR (400 MHz, CDCl₃) δ 8.50 (d, J=4.4 Hz, 1H), 7.36-7.33 (m, 2H), 7.09-7.02 (m, 2H), 6.46 (d, J=7.3 Hz, 1H), 4.19 (s, 2H), 3.95 (m, 1H), 3.85 (s, 2H), 3.22-3.16 (m, 2H), 3.01-2.78 (m, 5H), 2.67 (m, 1H), 2.53-2.46 (m, 2H), 2.39 (s, 3H), 2.28 (s, 3H), 2.13 (m, 1H), 2.06-1.97 (m, 2H), 1.67 (m, 1H); MS m/z 420 (M+1).

### Example 25

### N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}acetamide

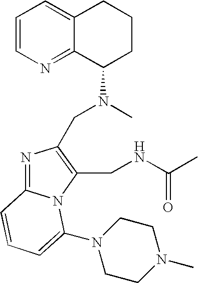

A solution of (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine (40 mg, 0.095 mmol) in dichloromethane (1 mL) was treated with N,N-diisopropylethylamine (33 μL, 0.19 mmol) and acetyl chloride (7.5 μL, 0.105 mmol). The reaction was stirred at room temperature for 15 hours and added directly to a silica column for purification by flash chromatography (0-10% ammonium hydroxide in acetonitrile) to give 22 mg (50% yield) N-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}acetamide as a tan solid. ¹H NMR (300 MHz, CDCl₃) δ 9.04 (br, 1H), 8.45 (d, J=4.4 Hz, 1H), 7.44-7.38 (m, 2H), 7.20-7.11 (m, 2H), 6.58 (d, J=7.2 Hz, 1H), 5.42 (br, 1H), 4.92 (m, 1H), 4.26-4.17 (m, 3H), 3.34-2.73 (m, 6H), 2.61 (s, 3H), 2.51-2.41 (m, 4H), 2.17-2.12 (m, 2H), 2.10 (s, 3H), 2.04 (s, 3H), 1.85-1.74 (m, 2H); MS m/z 462 (M+1).

### Example 26

### N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}propanamide

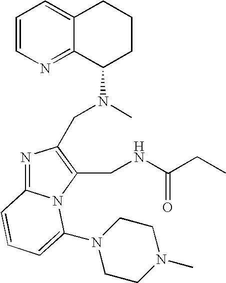

N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}propanamide was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and propionyl chloride in a similar manner as described herein to give an off-white solid (79% yield). ¹H NMR (400 MHz, CDCl₃) δ 8.82 (br, 1H), 8.36 (d, J=4.5 Hz, 1H), 7.34-7.30 (m, 2H), 7.09-7.05 (m, 1H), 7.03 (dd, J=7.6, 4.7 Hz, 1H), 6.45 (d, J=7.2 Hz, 1H), 5.49 (dd, J=15.6, 5.8 Hz, 1H), 4.79 (dd, J=15.5, 4.0 Hz, 1H), 4.05-3.99 (m, 3H), 3.29-3.18 (m, 2H), 3.02-2.86 (m, 4H), 2.81-2.64 (m, 4H), 2.46 (s, 3H), 2.33-2.25 (m, 6H), 2.05-1.97 (m, 2H), 1.69 (m, 1H), 1.13 (t, J=7.6 Hz, 3H); MS m/z 476 (M+1).

### Example 27

### 2-Methyl-N-{[5-(4-methyl-1-piperazinyl)-2-({methyl[(8S)-5,6,7,8-tetrahydro-8-quinolinyl]amino}methyl)imidazo[1,2-a]pyridine-3-yl]methyl}propanamide

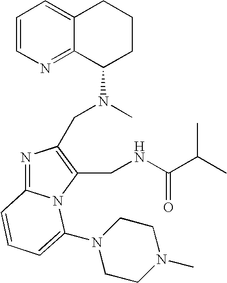

2-Methyl-N-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}propanamide was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and isobutyrl chloride in a similar manner as described herein to give a tan solid (83% yield). ¹H NMR (400 MHz, CDCl₃) δ 8.71 (br, 1H), 8.35 (d, J=4.4 Hz, 1H), 7.33-7.29 (m, 2H), 7.09-7.05 (m, 1H), 7.02 (dd, J=7.7; 4.7 Hz, 1H), 6.44 (d, J=7.2 Hz, 1H), 5.51 (dd, J=15.4, 5.8 Hz, 1H), 4.73 (dd, J=15.6, 3.7 Hz, 1H), 4.03-3.97 (m, 3H), 3.30-3.17 (m, 2H), 3.01-2.76 (m, 5H), 2.72-2.54 (m, 4H), 2.44 (s, 3H), 2.29-2.25 (m, 4H), 2.05-1.97 (m, 2H), 1.69 (m, 1H), 1.14-1.08 (m, 6H); MS m/z 490 (M+1).

### Example 28

### 2-Methyl-N-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}butanamide

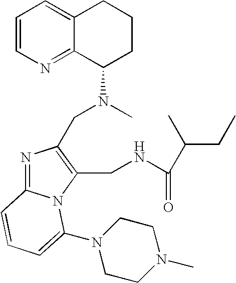

2-Methyl-N-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}butanamide was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and DL-2-methylbutyryl chloride in a similar manner as described herein to give an off-white solid (85% yield). ¹H NMR (400 MHz, CDCl₃) δ 8.63 (m, 1H), 8.34 (m, 1H), 7.32-7.28 (m, 2H), 7.08-7.04 (m, 1H), 7.00 (dd, J=7.9, 4.7 Hz, 1H), 6.43 (d, J=7.2 Hz, 1H), 5.51 (td, J=15.8, 5.8 Hz, 1H), 4.74 (m, 1H), 4.01-3.97 (m, 3H), 3.31-3.17 (m, 2H), 2.98 (m, 1H), 2.91-2.75 (m, 4H), 2.70-2.62 (m, 3H), 2.43 (s, 3H), 2.37-2.23 (m, 5H), 2.03-1.97 (m, 2H), 1.72-1.60 (m, 2H), 1.36 (m, 1H), 1.11-1.06 (m, 3H), 0.82-0.77 (m, 3H); MS m/z 504 (M+1).

### Example 29

### 2 N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}benzamide

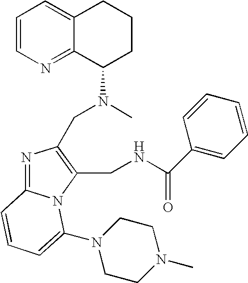

2 N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}benzamide was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and benzoyl chloride in a similar manner as described herein to give an off-white solid (81% yield). ¹H NMR (400 MHz, CDCl₃) δ 9.37 (s, 1H), 8.05 (s, 1H), 7.88 (d, J=7.7 Hz, 2H), 7.40-7.26 (m, 5H), 7.08 (m, 1H), 6.93 (m, 1H), 6.49 (d, J=7.1 Hz, 1H), 5.56 (m, 1H), 5.12 (m, 1H), 4.06-3.97 (m, 3H), 3.32-3.23 (m, 2H), 3.06-2.88 (m, 4H), 2.82-2.72 (m, 3H), 2.63 (m, 1H), 2.45 (s, 3H), 2.26 (s, 3H), 2.18 (m, 1H), 2.04-1.95 (m, 2H), 1.65 (m, 1H); MS m/z 524 (M+1).

### Example 30

### N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-2-phenylacetamide

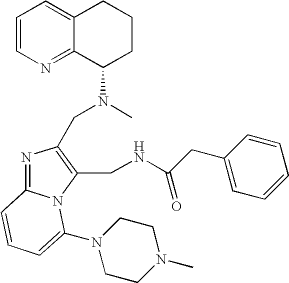

N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-2-phenylacetamide was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and phenylacetyl chloride in a similar manner as described herein to give a tan solid (74% yield). ¹H NMR (400 MHz, CDCl₃) δ 8.39 (d, J=4.2 Hz, 1H), 8.28 (br, 1H), 7.36-7.32 (m, 2H), 7.22-7.17 (m, 5H), 7.09-7:05 (m, 1H), 7.02 (dd, J=7.7, 4.7 Hz, 1H), 6.39 (d, J=7.1 Hz, 1H), 5.43 (dd, J=15.5, 6.2 Hz, 1H), 4.69 (dd, J=15.6, 4.1 Hz, 1H), 4.03-3.96 (m, 3H), 3.60 (s, 2H), 3.07 (m, 1H), 2.97-2.62 (m, 7H), 2.39-2.35 (m, 2H), 2.32 (s, 3H), 2.27 (s, 3H), 2.06-1.98 (m, 3H), 1.70 (m, 1H); MS m/z 538 (M+1).

### Example 31

### Methyl {[5-(4-methyl-1-piperazinyl)-2-({methyl[(8S)-5,6,7,8-tetrahydro-8-quinolinyl]amino}methyl)imidazo[1,2-a]pyridin-3-yl]methyl}carbamate

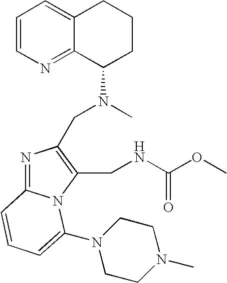

A solution of (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine (50 mg, 0.12 mmol) in dichloromethane (1 mL) was treated with N,N-diisopropylethylamine (42 μL, 0.24 mmol) and methyl chloroformate (11 μL, 0.14 mmol). The reaction was stirred at room temperature for 15 hours and added directly to a silica column for purification by flash chromatography (0-10% ammonium hydroxide in acetonitrile) to give 43 mg (75% yield) methyl {[5-(4-methyl-1-piperazinyl)-2-({methyl[(8S)-5,6,7,8-tetrahydro-8-quinolinyl]amino}methyl)imidazo[1,2-a]pyridin-3-yl]methyl}carbamate as an off-white solid. ¹H NMR (400 MHz, CDCl₃) δ 8.44 (s, 1H), 7.93 (br, 1H), 7.33-7.29 (m, 2H), 7.07 (m, 1H), 7.01 (m, 1H), 6.48 (d, J=7.4 Hz, 1H), 5.19 (dd, J=15.0, 7.2 Hz, 1H), 4.76 (d, J=14.3 Hz, 1H), 3.99-3.89 (m, 3H), 3.63 (s, 3H), 3.23-3.17 (m, 2H), 3.02-2.60 (m, 8H), 2.45 (s, 3H), 2.36 (m, 1H), 2.23 (s, 3H), 2.02-1.93 (m, 2H), 1.65 (m, 1H); MS m/z 478 (M+1).

### Example 32

### Ethyl {[5-(4-methyl-1-piperazinyl)-2-({methyl[(8S)-5,6,7,8-tetrahydro-8-quinolinyl]amino}methyl)imidazo[1,2-a]pyridin-3-yl]methyl}carbamate

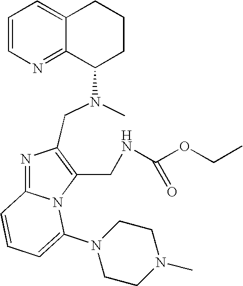

Ethyl {[5-(4-methyl-1-piperazinyl)-2-({methyl[(8S)-5,6,7,8-tetrahydro-8-quinolinyl]amino}methyl)imidazo[1,2-a]pyridin-3-yl]methyl}carbamate was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and ethyl chloroformate in a similar manner as described herein to give a tan solid (78% yield). ¹H NMR (400 MHz, CDCl₃) δ 8.49 (s, 1H), 7.82 (br, 1H), 7.34-7.30 (m, 2H), 7.10-7.06 (m, 1H), 7.02-6.99 (m, 1H), 6.48 (d, J=7.3 Hz, 1H), 5.19 (dd, J=14.9, 7.1 Hz, 1H), 4.77 (d, J=14.1 Hz, 1H), 4.11-4.05 (m, 2H), 4.01-3.89 (m, 3H), 3.25-3.18 (m, 2H), 3.02-2.74 (m, 6H), 2.70-2.61 (m, 2H), 2.45 (s, 3H), 2.27-2.20 (m, 4H), 2.03-1.96 (m, 2H), 1.67 (m, 1H), 1.25-1.18 (m, 3H); MS m/z 492 (M+1).

### Example 33

### Phenylmethyl {[5-(4-methyl-1-piperazinyl)-2-({methyl[(8S)-5,6,7,8-tetrahydro-8-quinolinyl]amino}methyl)imidazor[1,2-a]pyridin-3-yl]methyl}carbamate

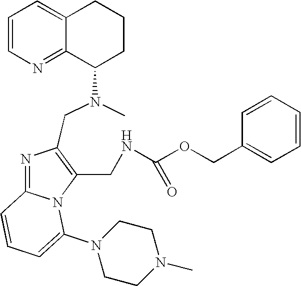

Phenylmethyl {[5-(4-methyl-1-piperazinyl)-2-({methyl[(8S)-5,6,7,8-tetrahydro-8-quinolinyl]amino}methyl)imidazo[1,2-a]pyridin-3-yl]methyl}carbamate was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and benzyl chloroformate in a similar manner as described herein to give an off-white solid (26% yield). ¹H NMR (400 MHz, CDCl₃) δ 8.30 (s, 1H), 7.35-7.24 (m, 6H), 7.15-7.05 (m, 2H), 6.85 (m, 1H), 6.48 (m, 1H), 5.24 (dd, J=14.8, 7.4 Hz, 1H), 5.14 (d, J=12.6 Hz, 1H), 5.06 (d, J=12.3 Hz, 1H), 4.77 (dd, J=14.7, 3.3 Hz, 1H), 4.01-3.92 (m, 2H), 3.85 (m, 1H), 3.20-3.12 (m, 2H), 3.03-2.56 (m, 8H), 2.42 (s, 3H), 2.32 (m, 1H), 2.20 (s, 3H), 1.99-1.90 (m, 2H), 1.62 (m, 1H), MS m/z 554 (M+1).

### Example 34

### N-{[5-(4-Methyl-1-piperazinyl)-2-({methyl[(8S)-5,6,7,8-tetrahydro-8-quinolinyl]amino}methyl)imidazo[1,2-a]pyridine-3-yl]methyl}methanesulfonamide

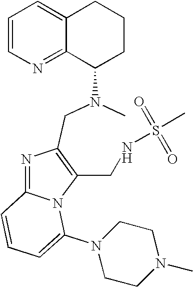

A solution of (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine (40 mg, 0.095 mmol) in dichloromethane (1 mL) was treated with N,N-diisopropylethylamine (33 μL, 0.19 mmol) and methane sulfonyl chloride (8.1 μL, 0.105 mmol). The reaction was stirred at room temperature for 15 hours and added directly to a silica column for purification by flash chromatography (0-10% ammonium hydroxide in acetonitrile) to give 13 mg (28% yield) N-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}methanesulfonamide as an off-white solid. ¹H NMR (300 MHz, CDCl₃) δ 8.83 (br, 1H), 8.68 (d, J=4.6 Hz, 1H), 7.41 (d, J=8.7 Hz, 1H), 7.36 (d, J=7.6 Hz, 1H), 7.22-7.16 (m, 1H), 7.06 (dd, J=7.7, 4.8 Hz, 1H), 6.62 (d, J=7.0 Hz, 1H), 5.29 (d, J=12.9 Hz, 1H), 4.83 (d, J=13.1 Hz, 1H), 4.03 (dd, J=29.7, 13.2 Hz, 2H), 3.83 (m, 1H), 3.37 (m, 1H), 3.22 (m, 1H), 3.11-2.96 (m, 4H), 2.93 (s, 3H), 2.82-2.65 (m, 2H), 2.52 (s, 3H), 2.37-2.28 (m, 2H), 2.24 (s, 3H), 2.09-1.92 (m, 3H), 1.71 (m, 1H); MS m/z 498 (M+1).

### Example 35

### N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}ethanesulfonamide

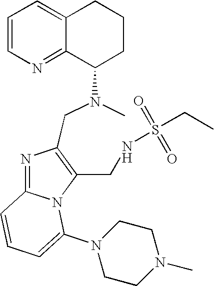

N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}ethanesulfonamide was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and ethane sulfonyl chloride in a similar manner as described herein to give an off-white solid (80% yield). ¹H NMR (400 MHz, CDCl₃) δ 8.72 (br, 1H), 8.60 (d, J=4.8 Hz, 1H), 7.34 (d, J=8.7 Hz, 1H), 7.30 (d, J=7.7 Hz, 1H), 7.15-7.11 (m, 1H), 6.99 (dd, J=7.8, 4.9 Hz, 1H), 6.53 (d, J=7.1 Hz, 1H), 5.21 (d, J=13.6 Hz, 1H), 4.86 (d, J=13.8 Hz, 1H), 3.97 (dd, J=33.1, 13.0 Hz, 2H), 3.81 (m, 1H), 3.28 (m, 1H), 3.16 (m, 1H), 3.02-2.72 (m, 8H), 2.66-2.60 (m, 2H), 2.45 (s, 3H), 2.27 (m, 1H), 2.18 (s, 3H), 2.01-1.87 (m, 2H), 1.66 (m, 1H), 1.21 (t, J=7.5 Hz, 3H); MS m/z 512 (M+1).

### Example 36

### N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-2-propanesulfonamide

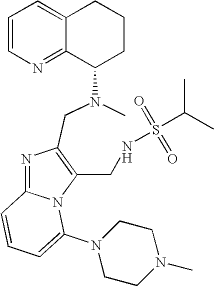

N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-2-propanesulfonamide was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and isopropyl sulfonyl chloride in a similar manner as described herein to give an off-white solid (46% yield). ¹H NMR (400 MHz, CDCl₃) δ 8.66 (br, 1H), 8.59 (d, J=4.5 Hz, 1H), 7.33 (d, J=8.9 Hz, 1H), 7.30 (d, J=7.9 Hz, 1H), 7.14-7.10 (m, 1H), 6.99 (dd, J=7.7, 4.9 Hz, 1H), 6.50 (d, J=7.2 Hz, 1H), 5.19 (d, J=14.3 Hz, 1H), 4.97 (d, J=14.4 Hz, 1H), 3.97 (dd, J=28.1, 12.8 Hz, 2H), 3.87 (m, 1H), 3.25-3.16 (m, 2H), 3.09-2.95 (m, 2H), 2.91-2.72 (m, 6H), 2.64 (m, 1H), 2.45 (s, 3H), 2.25 (m, 1H), 2.18 (s, 3H), 2.01-1.89 (m, 2H), 1.67 (m, 1H), 1.22-1.19 (m, 6H); MS m/z 526 (M+1).

### Example 37

### N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}benzenesulfonamide

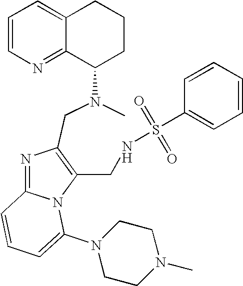

N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}benzenesulfonamide was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and benzene sulfonyl chloride in a similar manner as described herein to give a pale yellow solid (79% yield). ¹H NMR (400 MHz, CDCl₃) δ 9.22 (br, 1H), 8.75 (d, J=4.5 Hz, 1H), 7.76 (d, J=7.8 Hz, 2H), 7.38 (m, 1H), 7.31-7.25 (m, 4H), 7.10-7.06 (m, 1H), 7.02 (dd, J=7.6, 4.8 Hz, 1H), 6.46 (d, J=7.2 Hz, 1H), 5.17 (d, J=13.3 Hz, 1H), 4.54 (d, J=13.3 Hz, 1H), 3.83 (d, J=13.2 Hz, 1H), 3.70 (m, 1H), 3.65 (d, J=12.9 Hz, 1H), 3.22-3.10 (m, 2H), 3.01-2.91 (m, 2H), 2.85-2.70 (m, 5H), 2.62 (m, 1H), 2.47 (s, 3H), 2.22 (m, 1H), 1.89 (s, 3H), 1.86-1.80 (m, 2H), 1.63 (m, 1H); MS m/z 560 (M+1).

### Example 38

### N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-1-phenylmethanesulfonamide

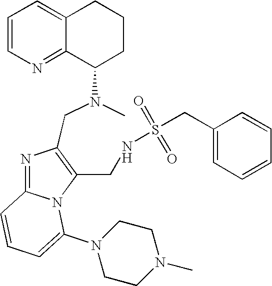

N-{[5-(4-Methyl-1-piperazinyl)-2-({methyl[(8S-5,6,7,8-tetrahydro-8-quinolinyl]amino}methyl)imidazo[1,2-a]pyridin-3-yl]methyl}-1-phenylmethanesulfonamide was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and benzyl sulfonyl chloride in a similar manner as described herein to give a white solid (39% yield). ¹H NMR (400 MHz, CDCl₃) δ 8.85 (br, 1H), 8.56 (d, J=4.4 Hz, 1H), 7.39 (d, J=8.7 Hz, 1H), 7.31 (d, J=7.5 Hz, 1H), 7.26 (m, 1H), 7.17-7.11 (m, 3H), 7.05-7.00 (m, 3H), 6.53 (d, J=7.2 Hz, 1H), 5.02 (d, J=13.6 Hz, 1H), 4.84 (d, J=13.9 Hz, 1H), 4.17 (s, 2H), 3.96 (dd, J=32.7, 13.0 Hz, 2H), 3.80 (m, 1H), 3.18-3.07 (m, 2H), 3.01-2.73 (m, 7H), 2.65 (m, 1H), 2.43 (s, 3H), 2.26 (m, 1H), 2.12 (s, 3H), 2.02 (m, 1H), 1.90 (m, 1H), 1.66 (m, 1H); MS m/z 574 (M+1).

### Example 39

### N-Ethyl-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea

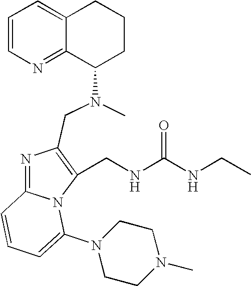

A solution of (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine (50 mg, 0.12 mmol) in dichloromethane (1 mL) was treated with ethyl isocyanate (11 μL, 0.14 mmol). The reaction was stirred at room temperature for 15 hours and added directly to a silica column for purification by flash chromatography (0-10% ammonium hydroxide in acetonitrile) to give 51 mg (86% yield) N-ethyl-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea as an off-white solid. ¹H NMR (400 MHz, CDCl₃) δ 8.38 (d, J=4.4 Hz, 1H), 7.78 (br, 1H), 7.38 (d, J=7.5 Hz, 1H), 7.27 (d, J=8.9 Hz, 1H), 7.10-7.03 (m, 2H), 6.95 (br, 1H), 6.39 (d, J=7.3 Hz, 1H), 5.12 (s, 2H), 4.11 (m, 1H), 3.93 (dd, J=44.8, 12.6 Hz, 2H), 3.29-3.16 (m, 4H), 2.89-2.63 (m, 8H), 2.40 (s, 3H), 2.29 (m, 1H), 2.04 (s, 3H), 2.01-1.92 (m, 2H), 1.70 (m, 1H), 1.02 (t, J=7.3 Hz, 3H); MS m/z 491 (M+1).

### Example 40

### N-(1-Methylethyl)-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea

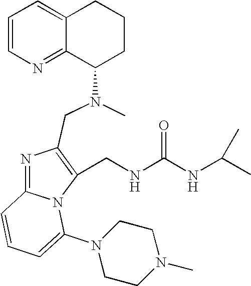

N-(1-Methylethyl)-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and isopropyl isocyanate in a similar manner as described herein to give a white solid (56% yield). ¹H NMR (400 MHz, CDCl₃) δ 8.44 (d, J=4.3 Hz, 1H), 8.32 (br, 1H), 7.39 (d, J=7.6 Hz, 1H), 7.28 (d, J=8.4 Hz, 1H), 7.10-7.03 (m, 2H), 6.51 (br, 1H), 6.38 (d, J=7.1 Hz, 1H), 5.25 (d, J=15.5 Hz, 1H), 5.06 (d, J=15.5 Hz, 1H), 4.10 (m, 1H), 4.03-3.97 (m, 2H), 3.86 (m, 1H), 3.31-3.22 (m, 2H), 2.91-2.59 (m, 8H), 2.41 (s, 3H), 2.30 (m, 1H), 2.02 (s, 3H), 1.97-1.93 (m, 2H), 1.71 (m, 1H), 1.07 (d, J=6.7 Hz, 3H), 1.01 (d, J=6.5 Hz, 3H); MS m/z 505 (M+1).

### Example 41

### N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-N′-phenylurea

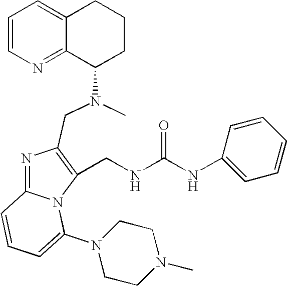

N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-N′-phenylurea was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and phenyl isocyanate in a similar manner as described herein to give an off-white solid (80% yield). ¹H NMR (400 MHz, CDCl₃) δ 9.31 (s, 1H), 8.80 (br, 1H), 8.47 (d, J=4.2 Hz, 1H), 7.44 (d, J=7.5 Hz, 1H), 7.34-7.28 (m, 3H), 7.20-7.12 (m, 3H), 7.09-7.05 (m, 1H), 6.91-6.88 (m, 1H), 6.42 (d, J=7.1 Hz, 1H), 5.45 (br, 1H), 5.07 (m, 1H), 4:18 (m, 1H), 4.10 (m, 1H), 3.94 (d, J=12.5 Hz, 1H), 3.32-3.20 (m, 2H), 2.95-2.78 (m, 4H), 2.75-2.59 (m, 4H), 2.42 (s, 3H), 2.31 (m, 1H), 2.05 (s, 3H), 2.02-1.96 (m, 2H), 1.73 (m, 1H); MS m/z 539 (M+1).

### Example 42

### N-[4-(Dimethylamino)phenyl]-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea

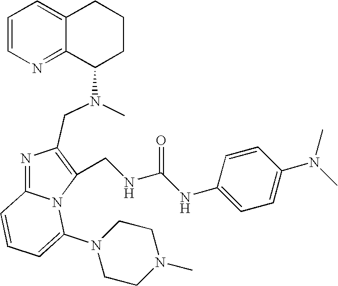

N-[4(Dimethylamino)phenyl]-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and 4-(dimethylamino)phenyl isocyanate in a similar manner as described herein to give a pale yellow solid (84% yield). ¹H NMR (400 MHz, CDCl₃) δ 8.93 (br, 1H), 8.46 (d, J=4.3 Hz, 1H), 8.37 (br, 1H), 7.41 (d, J=7.4 Hz, 1H), 7.29 (d, J=8.8 Hz, 1H), 7.19-7.17 (m, 2H), 7.12-7.05 (m, 2H), 6.66-6.63 (m, 2H), 6.41 (d, J=7.0 Hz, 1H), 5.33 (m, 1H), 5.12 (m, 1H), 4.18 (m, 1H), 4.08 (m, 1H), 3.94 (d, J=12.3 Hz, 1H), 3.30-3.19 (m, 2H), 2.92-2.59 (m, 14H), 2.40 (s, 3H), 2.29 (m, 1H), 2.05 (s, 3H), 2.01-1.96 (m, 2H), 1.71 (m, 1H); MS m/z 582 (M+1).

### Example 43

### N-[4-(Methyloxy)phenyl]-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea

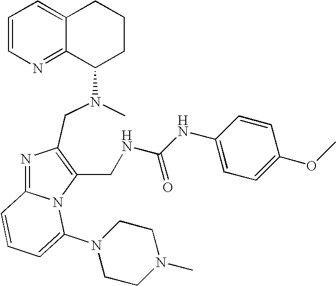

N-[4-(Methyloxy)phenyl]-N′-{[5-(4-methyl-1-piperazinyl)-2-({methyl[(8S-5,6,7,8-tetrahydro-8-quinolinyl]amino}methyl)imidazo[1,2-a]pyridin-3-yl]methyl}urea was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-8-quinolinamine and 4-methoxyphenyl isocyanate in a similar manner as described herein to give an off-white solid (59% yield). ¹H NMR (400 MHz, CDCl₃) δ 9.22 (br, 1H), 8.60 (br, 1H), 8.45 (s, 1H), 7.43 (d, J=7.6 Hz, 1H), 7.31-7.22 (m, 3H), 7.14-7.08 (m, 2H), 6.77-6.73 (m, 2H), 6.43 (d, J=6.9 Hz, 1H), 5.36 (m, 1H), 5.13 (m, 1H), 4.20 (m, 1H), 4.10 (m, 1H), 3.96 (m, 1H), 3.73 (s, 3H), 3.31-3.20 (m, 2H), 2.92-2.59 (m, 8H), 2.41 (s, 3H), 2.31 (m, 1H), 2.06 (s, 3H), 2.02-1.96 (m, 2H), 1.71 (m, 1H); MS m/z 569 (M+1).

### Example 44

### N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}urea

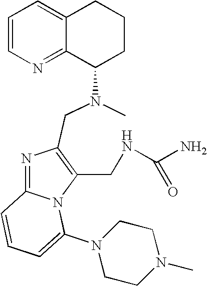

N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}urea was prepared from (8S)—N-{[3-(aminomethyl)-5-(4-methyl-1-piperazinyl)imidazo[1,2-a]pyridin-2-yl]methyl}-N-methyl-5,6,7,8-tetrahydro-quinolinamine and trimethylsilyl isocyanate in a similar manner as described herein to give a tan solid (75% yield). ¹H NMR (400 MHz, CDCl₃) δ 8.45 (br, 1H), 8.35 (s, 1H), 7.37 (d, J=7.7 Hz, 1H), 7.29 (d, J=8.9 Hz, 1H), 7.09-7.04 (m, 2H), 6.42 (d, J=6.9 Hz, 1H), 5.60 (br, 2H), 5.16 (s, 2H), 4.14 (m, 1H), 4.04-3.90 (m, 2H), 3.27-3.20 (m, 2H), 2.92-2.64 (m, 8H), 2.40 (s, 3H), 2.31 (m, 1H), 2.06 (s, 3H), 2.00-1.91 (m, 2H), 1.69 (m, 1H); MS m/z 463 (M+1).

### BIOLOGICAL SECTION

### HOS HIV-1 Infectivity Assay

### HIV Virus Preparation

Compounds were profiled against two HIV-1 viruses, the M-tropic (CCR5 utilizing) Ba-L strain and the T-tropic (CXCR4 utilizing) IIIB strain. Both viruses were propagated in human peripheral blood lymphocytes. Compounds were tested for there ability to block infection of the HOS cell line (expressing hCXCR4/hCCR5/hCD4/pHIV-LTR-luciferase) by either HIV-1 Ba-L or HIV-1 IIIB. Compound cytotoxicity was also examined in the absence of virus addition.

### HOS HIV-1 Infectivity Assay Format

HOS cells (expressing hCXCR4/hCCR5/hCD4/pHIV-LTR-luciferase) were harvested and diluted in Dulbeccos modified Eagles media supplemented with 2% FCS and non-essential amino acid to a concentration of 60,000 cells/ml. The cells were plated into 96-well plates (100 ul per well) and the plates were placed in a tissue culture incubator (37° C.; 5% CO₂/95% air) for a period of 24h.

Subsequently, 50 ul of the desired drug solution (4 times the final concentration) was added to each well and the plates were returned to the tissue culture incubator (37° C.; 5% CO₂/95% air) for 1h. Following this incubation 50 ul of diluted virus was added to each well (approximately 2 million RLU per well of virus). The plates were returned to the tissue culture incubator (37° C.; 5% CO₂/95% air) and were incubated for a further 96h.

Following this incubation the endpoint for the virally infected cultures was quantified following addition of Steady-Glo Luciferase assay system reagent (Promega, Madison, Wis.). Cell viability or non-infected cultures was measured using a CellTiter-Glo luminescent cell viability assay system (Promega, Madison, Wis.). All luminescent readouts are performed on a Topcount luminescence detector (Packard, Meridien, Conn.).

TABLE 1

| Ex- | | Ac- |
|---|---|---|
| am- | | tivity |
| ple | Structure | Level* |
| 10 | 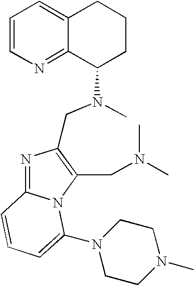 | A |
| 11 | 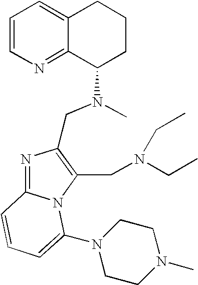 | A |
| 12 | 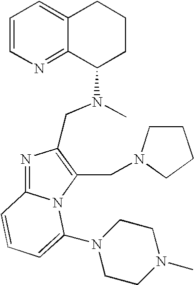 | A |
| 13 | 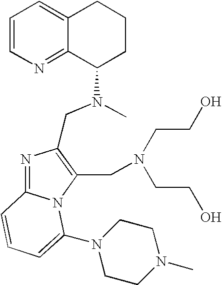 | A |
| 14 | 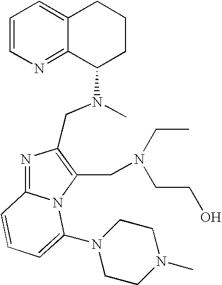 | A |
| 15 | 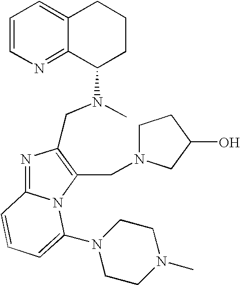 | A |
| 16 | 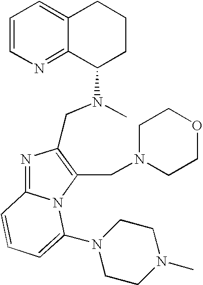 | A |
| 19 | 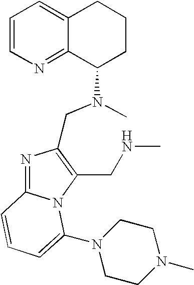 | A |
| 20 | 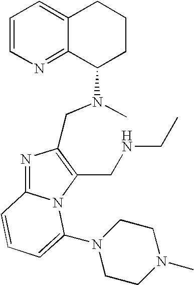 | A |
| 21 | 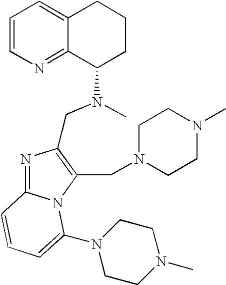 | A |
| 22 | 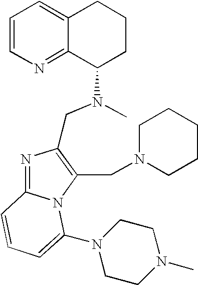 | A |
| 23 | 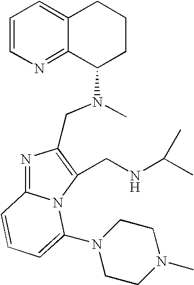 | A |
| 24 | 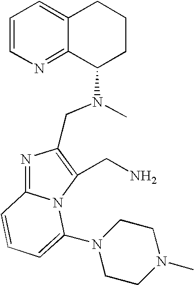 | A |
| 25 | 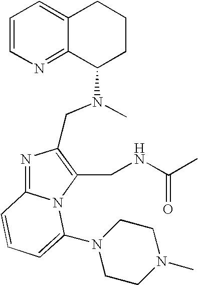 | A |
| 26 | 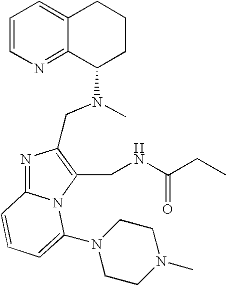 | A |
| 27 | 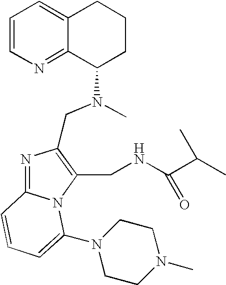 | A |
| 28 | 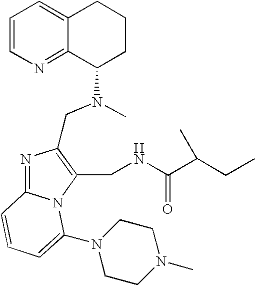 | A |
| 29 | 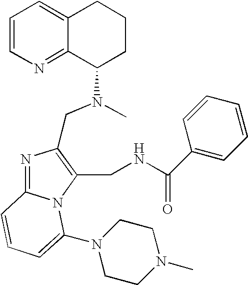 | A |
| 30 | 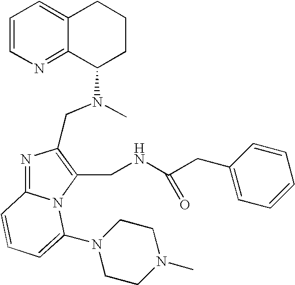 | A |
| 31 | 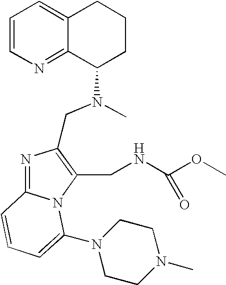 | A |
| 32 | 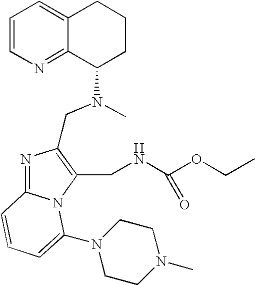 | A |
| 33 | 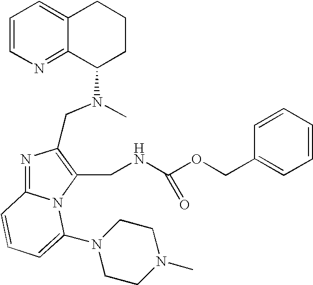 | A |
| 34 | 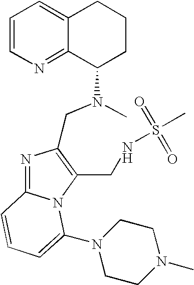 | A |
| 35 | 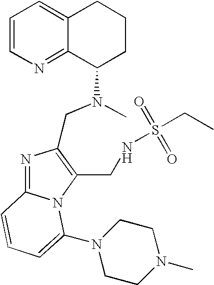 | A |
| 36 | 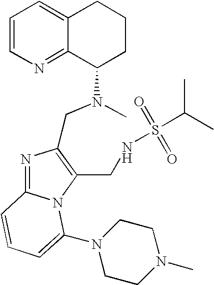 | A |
| 37 | 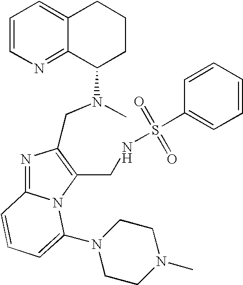 | A |
| 38 | 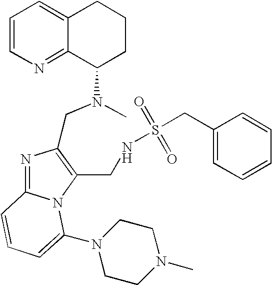 | A |
| 39 | 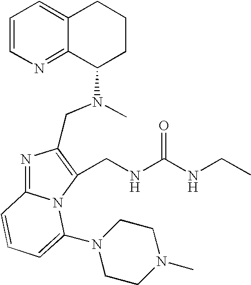 | A |
| 40 | 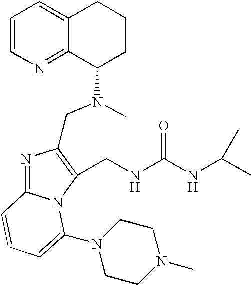 | A |
| 41 | 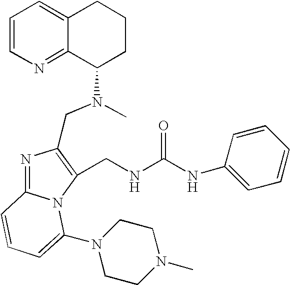 | A |
| 42 | 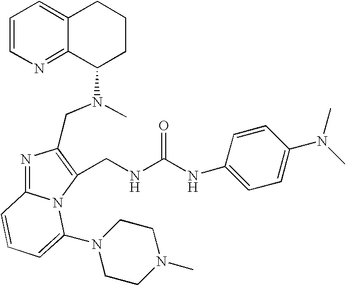 | A |
| 43 | 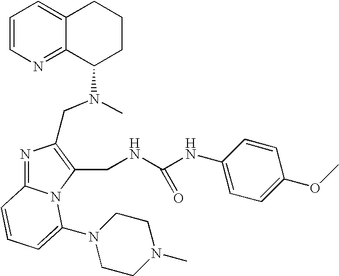 | A |
| 44 | 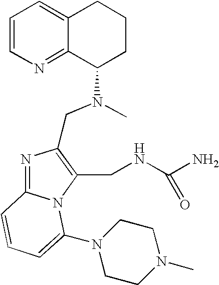 | A |

*“A” indicates an activity level of less than 100 nM in the HIV infectivity assay.
“B” indicates an activity level of between 100 nM to 500 nM in the HIV infectivity assay.
“C” indicates an activity level of between 500 nM and 10 μM in the HIV infectivity assay.

Compounds of the present invention demonstrate anti-HIV activity in the range of IC₅₀ of about 1 nM to about 50 μM. In one aspect of the invention, compounds of the present invention have anti-HIV activity in the range of up to about 100 nM. In another aspect of the invention, compounds of the present invention have anti-HIV activity in the range of from about 100 nM to about 500 nM. In another aspect of the invention, compounds of the present invention have anti-HIV activity in the range of from about 500 nM to 10 μM. In another aspect of the invention, compounds have anti-HIV activity in the range of from about 10 μM to about 50 μM. Moreover, compounds of the present invention are believed to provide a desired pharamcokinetic profile. Also, compounds of the present invention are believed to provide a desired selectivity, such as specificity between toxicity and activity.

Test compounds were employed in free or salt form.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of formula (I) 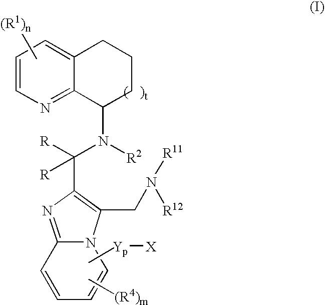
wherein:
t is 0, 1, or 2;
each R independently is H, C₁-C₈ alkyl, C₂-C₆ alkenyl, C₂-C₆ alkynyl, C₁-C₈ haloalkyl, C₃-C₈ cycloalkyl, —R^{a}Ay, —R^{a}OR¹⁰, or —R^{a}S(O)_{q}R¹⁰;
each R¹ independently is halogen, C₁-C₈ haloalkyl, C₁-C₈ alkyl, C₂-C₆ alkenyl, C₂-C₆ alkynyl, C₃-C₈ cycloalkyl, C₃-C₈ cycloalkenyl, -Ay, —NHAy, -Het, —NHHet, —OR¹⁰, —OAy, —OHet, R^{a}OR¹⁰, —NR⁶R⁷, —R^{a}NR⁶R⁷, R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from the group consisting of H, C₁-C₈ alkyl, C₁-C₈ haloalkyl, C₃-C₈ cycloalkyl, C₂-C₆ alkenyl, C₂-C₆ alkynyl, —R^{a}Ay, —R^{a}cycloalkyl, —R^{a}OR⁵, and —R^{a}S(O)_{q}R⁵;
each R⁴ independently is halogen, C₁-C₈ haloalkyl, C₁-C₈ alkyl, C₂-C₆ alkenyl, C₂-C₆ alkynyl, C₃-C₈ cycloalkyl, C₃-C₈ cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, C₁-C₈ alkyl, C₂-C₆ alkenyl, C₂-C₆ alkynyl, C₃-C₈ cycloalkyl, or -Ay;
p is 0 or 1;
Y is —NR¹⁰—, —O—, —C(O)NR¹⁰—, —NR¹⁰C(O)—, —C(O)—, —C(O)O—, —NR¹⁰C(O)N(R¹⁰)—, —S(O)_{q}—, S(O)_{q}NR¹⁰ or —NR¹⁰S(O)_{q}—;
X is —N(R¹⁰)₂, —R^{a}N(R¹⁰)₂, AyN(R¹⁰)₂, R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is C₁-C₈ alkylene, C₃-C₈ cycloalkylene, C₂-C₆ alkenylene, C₃-C₈ cycloalkenylene, or C₂-C₆ alkynylene, optionally substituted with one or more of C₁-C₈ alkyl, hydroxyl or oxo;
each of R⁶ and R⁷ independently is selected from H, C₁-C₈ alkyl, C₂-C₆ alkenyl, C₂-C₆ alkynyl, C₃-C₇ cycloalkyl, C₃-C₇ cycloalkenyl, —R^{a}cycloalkyl, —R^{a}OH, —R^{a}OR⁵, —R^{a}NR⁸R⁹, -Ay, -Het, —R^{a}Ay, —R^{a}Het, or —S(O)_{q}R⁵;
each of R⁸ and R⁹ independently is selected from H or C₁-C₈ alkyl;
each R¹⁰ independently is H, C₁-C₈ alkyl, C₃-C₈ cycloalkyl, C₂-C₆ alkenyl, C₂-C₆ alkynyl, C₃-C₈ cycloalkenyl, —R^{a}cycloalkyl, —R^{a}OH, —R^{a}OR⁵, —R^{a}NR⁶R⁷, or —C(O)R^{a}Ay;
each of R¹¹ and R¹² independently is selected from the group consisting of H, C₁-C₈ alkyl, C₁-C₈ haloalkyl, C₂-C₆ alkenyl, C₂-C₆ alkynyl, C₃-C₈cycloalkyl, C₃-C₈ cycloalkenyl, -Ay, -Het, —R^{a}OR¹⁰, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, —S(O)_{q}Het, —CO₂R^{a}Ay, —S(O)_{q}R^{a}Ay, and —R^{a}Het; or R¹¹ and R¹² link to form a heterocyclic ring optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino;
each q independently is 0, 1, or 2;
each Ay independently represents a C₄-C₁₄ aryl group optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; and
each Het independently represents a C₃-C₁₁ heterocyclyl or heteroaryl group, optionally substituted with one or more of C₁-C₈ alkyl, C₂-C₆ alkenyl, C₂-C₆ alkynyl, C₁-C₈ alkoxy, hydroxyl, halogen, C₁-C₈ haloalkyl, C₃-C₈ cycloalkyl, C₃-C₈ cycloalkoxy, cyano, amide, amino, and C₁-C₈ alkylamino; or pharmaceutically acceptable derivates thereof.

2. The compound of claim 1 wherein t is 1.

3. (canceled)

4. The compound of claim 3 wherein each R is H.

5. The compound of claim 1 wherein n is 0.

6-8. (canceled)

9. The compound of claim 1 wherein m is 0.

10-14. (canceled)

15. The compound of claim 14 wherein X is —R^{a}N(R¹⁰)₂, -Het, —R^{a}Het, or -HetN(R¹⁰)₂.

16. The compound of claim 1 wherein
p is 1;
Y is —N(R¹⁰)—, —O—, —S—, —CONR¹⁰—, —NR¹⁰CO—, or —S(O)_{q}NR¹⁰—; and
X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂.

17. The compound of claim 16 wherein
Y is —N(R¹⁰)—, —O—, —CONR¹⁰—, or —NR¹⁰CO—; and
X is —R^{a}N(R¹⁰)₂, -Het, —R^{a}Het, or -HetN(R¹⁰)₂.

18. The compound of claim 1 wherein the substituent —Yₚ—X is located on the depicted benzimidazole ring as in formula (I-A): 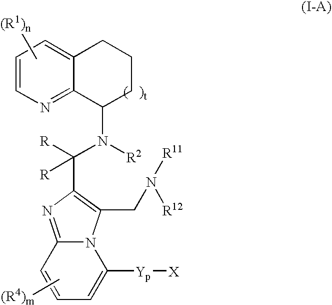
or pharmaceutically acceptable derivatives thereof.

19. The compound of claim 1 wherein Het is piperidine, piperazine, azetidine, pyrrolidine, imidazole, or pyridine.

20. The compound of claim 1 wherein p is 0 and X is -Het.

21. The compound of claim 20 wherein -Het is unsubstituted or substituted with one or more C₁-C₈ alkyl or C₃-C₈ cycloalkyl.

22. A compound selected from the group consisting of
(8S)—N-{[3-[(Dimethylamino)methyl]-5-(4-methyl-1-piperazinyl)imidazo[1,2-a]pyridin-2-yl]methyl}-N-methyl-5,6,7,8-tetrahydro-8-quinolinamine;
(8S)—N-{[3-[(Diethylamino)methyl]-5-(4-methyl-1-piperazinyl)imidazo[1,2-a]pyridin-2-yl]methyl}-N-methyl-5,6,7,8-tetrahydro-8-quinolinamine;
(8S)—N-Methyl-N-{[5-(4-methyl-1-piperazinyl)-3-(1-pyrrolidinylmethyl)imidazo[1,2-a]pyridin-2-yl]methyl}-5,6,7,8-tetrahydro-8-quinolinamine;
2,2′-({[5-(4-Methyl-1-piperazinyl)-2-({methyl[(8S)-5,6,7,8-tetrahydro-8-quinolinyl]amino}methyl)imidazo[1,2-a]pyridin-3-yl]methyl}imino)diethanol;
2-(Ethyl{[5-(4-methyl-1-piperazinyl)-2-({methyl[(8S)-5,6,7,8-tetrahydro-8-quinolinyl]amino}methyl)imidazo[1,2-a]pyridin-3-yl]methyl}amino)ethanol;
1-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-3-pyrrolidinol;
(8S)—N-Methyl-N-{[5-(4-methyl-1-piperazinyl)-3-(4-morpholinylmethyl)imidazo[1,2-a]pyridin-2-yl]methyl}-5,6,7,8-tetrahydro-8-quinolinamine;
(8S)—N-Methyl-N-{[3-[(methylamino)methyl]-5-(4-methyl-1-piperazinyl)imidazo[1,2-a]pyridin-2-yl]methyl}-5,6,7,8-tetrahydro-8-quinolinamine;
(8S)—N-{[3-[(Ethylamino)methyl]-5-(4-methyl-1-piperazinyl)imidazo[1,2-a]pyridin-2-yl]methyl}-N-methyl-5,6,7,8-tetrahydro-8-quinolinamine;
(8S)—N-Methyl-N-({5-(4-methyl-1-piperazinyl)-3-[(4-methyl-1-piperazinyl)methyl]imidazo[1,2-a]pyridin-2-yl}methyl)-5,6,7,8-tetrahydro-8-quinolinamine;
(8S)—N-Methyl-N-{[5-(4-methyl-1-piperazinyl)-3-(1-piperidinylmethyl)imidazo[1,2-a]pyridin-2-yl]methyl}-5,6,7,8-tetrahydro-8-quinolinamine;
(8S)—N-Methyl-N-{[3-{[(1-methylethyl)amino]methyl}-5-(4-methyl-1-piperazinyl)methyl]imidazo[1,2-a]pyridin-2-yl]methyl}-5,6,7,8-tetrahydro-8-quinolinamine;
(8S)—N-{[3-(Aminomethyl)-5-(4-methyl-1-piperazinyl)imidazo[1,2-a]pyridin-2-yl]methyl}-N-methyl-5,6,7,8-tetrahydro-8-quinolinamine;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}acetamide;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}propanamide;
2-Methyl-N-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}propanamide;
2-Methyl-N-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}butanamide;
2 N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}benzamide;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-2-phenylacetamide;
Methyl {[5-(4-methyl-1-piperazinyl)-2-({methyl[(8S)-5,6,7,8-tetrahydro-8-quinolinyl]amino}methyl)imidazo[1,2-a]pyridin-3-yl]methyl}carbamate;
Ethyl {[5-(4-methyl-1-piperazinyl)-2-({methyl[(8S)-5,6,7,8-tetrahydro-8-quinolinyl]amino}methyl)imidazo[1,2-a]pyridin-3-yl]methyl}carbamate;
Phenylmethyl {[5-(4-methyl-1-piperazinyl)-2-({methyl[(8S)-5,6,7,8-tetrahydro-8-quinolinyl]amino}methyl)imidazo[1,2-a]pyridin-3-yl]methyl}carbamate;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}methanesulfonamide;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}ethanesulfonamide;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-2-propanesulfonamide;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}benzenesulfonamide;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-1-phenylmethanesulfonamide;
N-Ethyl-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea;
N-(1-Methylethyl)-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}-N′-phenylurea;
N-[4-(Dimethylamino)phenyl]-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea;
N-[4-(Methyloxy)phenyl]-N′-{[5-(4-methyl-1-piperazinyl)-2-({methyl[(8S)-5,6,7,8-tetrahydro-8-quinolinyl]amino}methyl)imidazo[1,2-a]pyridin-3-yl]methyl}urea;
N-{[5-(4-Methyl-1-piperazinyl)-2-({methyl[(8S)-5,6,7,8-tetrahydro-8-quinolinyl]amino}methyl)imidazo[1,2-a]pyridin-3-yl]methyl}urea; and
pharmaceutically acceptable derivatives thereof.

23. (canceled)

24. A pharmaceutical composition comprising a compound according to claim 1, and a pharmaceutically acceptable carrier.

25-43. (canceled)

44. The compound of claim 1, wherein R² is C₁-C₈ alkyl.

45. A pharmaceutical composition comprising a compound according to claim 18, and a pharmaceutically acceptable carrier.

46. A pharmaceutical composition comprising a compound according to claim 22, and a pharmaceutically acceptable carrier.
